(19) United States
(12) Patent Application Publication
(10) Pub. No.: US 2018/0271423 A1
(43) Pub. Date: Sep. 27, 2018

(54) A BIOSENSOR DEVICE TO DETECT TARGET ANALYTES IN SITU, IN VIVO, AND/OR IN REAL TIME, AND METHODS OF MAKING AND USING THE SAME

(71) Applicant: The University of Toledo, Toledo, OH (US)

(72) Inventors: Anand K. Agarwal, Toledo, OH (US); Vijay K. Goel, Toledo, OH (US); Dong-Shik Kim, Toledo, OH (US); Boren Lin, Toledo, OH (US)

(73) Assignee: The University of Toledo, Toledo, OH (US)

(21) Appl. No.: 15/764,627

(22) PCT Filed: Oct. 6, 2016
(86) PCT No.: PCT/US2016/055706

§ 371 Date: Mar. 29, 2018

Related U.S. Application Data
(60) Provisional application No. 62/238,316, filed on Oct. 7, 2015.

(51) Int. Cl. A61B 5/145; A61B 5/1477; A61B 5/00; A61B 5/053

(52) U.S. Cl. CPC A61B 5/14546; A61B 5/1477; A61B 5/04; A61B 5/0537; A61B 5/7435

(57) ABSTRACT

A biosensor device for the real-time detection of a target analyte includes a receptor component operatively connected to a transducer component which is adapted to interpret and transmit a detectable signal. The receptor component includes a sensing element capable of detecting and binding to at least one target analyte, and a self-assembled monolayer (SAM) layer. The SAM layer is positioned between and in contact with the sensing element and an electrode such that the sensing element, in the presence of the target analyte, causes a detectable signal capable of being transmitted to the electrode. The SAM layer may include an anti-fouling agent. The transducer component includes an electrode (or set of electrodes that includes a working electrode) and microprocessor configured to screen noise and to pick up a detectable signal, such as impedance change at a very low frequency range.

### RELATED APPLICATIONS

This application claims priority to U.S. provisional patent application 62/238,316, filed Oct. 7, 2015, the entire disclosure of which is expressly incorporated herein by reference for all purposes.

### STATEMENT REGARDING FEDERALLY SPONSORED RESEARCH

This invention was not made with any government support. The government has no rights in this invention.

### TECHNICAL FIELD

The present disclosure pertains to the field of sensing target analytes in real time and in situ.

### BACKGROUND OF THE INVENTION

It is difficult to detect the presence of a target analyte directly either in a sample (e.g., in situ) or inside a body (e.g., in vivo). Currently, target analytes are detected by removing a sample and submitting such sample to a laboratory for analysis. It is also difficult to detect the target analyte in real time. Current techniques require a sample to be taken from a patient and then analyzed in a laboratory which greatly delays the timing of any diagnosis or detection of possibly toxic analytes. There is a need for a sensor with improved sensing characteristics and real-time in vivo/in situ detection capability. It would be further beneficial to have a sensor capable of rapid differentiation between viral and bacterial infections, and, if bacterial, segmentation into either Gram-positive or Gram-negative bacteria.

### SUMMARY OF THE INVENTION

Disclosed herein is a biosensor for detecting the presence of a target analyte.

In a first aspect, a biosensor includes: a transducer component comprising a set of electrodes operatively connected to a microprocessor, the microprocessor being adapted to receive, process and transmit a signal, and the set of electrodes including at least one working electrode; and, a receptor component having: i) a sensing element capable of binding to at least one target analyte present in a sample; and, ii) at least one anti-fouling agent linked to the working electrode; where the sensing element is attached to the working electrode either directly or via a self-assembled monolayer (SAM) layer positioned between and in contact with the sensing element and the working electrode. The transducer component and the receptor component are capable of being brought into direct contact with the sample in situ. In use, the sensing element, in the presence of target analyte present in the sample, causes a detectable signal capable of being transmitted to the working electrode. In particular embodiments, the SAM layer comprises 11-mercaptoundecanoic acid (11-MUA) and 3,6-dioxa-8-mercaptooctan-1-ol (DMOL). In certain embodiments, the anti-fouling agent comprises 3,6-dioxa-8-mercaptooctan-1-ol (DMOL) or PEG3.

In certain embodiments, the sensing element comprises one or more proteins, peptides, aptamers, nucleic acids, or polymers. In certain embodiments, the sensing element comprises one or more of an antibody, an enzyme, an antibody fragment, DNA, RNA, an aptamer, an oligonucleotide, or a synthetic or natural polymer. In certain embodiments, the sensing element comprises a polymer selected from the group consisting of alginate, chitosan, carboxymethyl cellulose, and derivatives thereof. In certain embodiments, the sensing element comprises at least one antibody capable of binding to at least one bacterial target analyte.

In certain embodiments, the biosensor is battery powered. In certain embodiments, the presence of the target analyte is detected in real time.

In certain embodiments, the sample comprises a fluid or tissue in a living organism. In certain embodiments, the sample comprises a fluid or tissue in a living organism in vivo. In certain embodiments, the sample comprises a fluid or tissue in a living animal. In certain embodiments, wherein the sample comprises a fluid or tissue in a human. In certain embodiments, the sample comprises a food product.

In certain embodiments, the rate and degree of signal change correspond to the presence and concentration of the target analyte. In certain embodiments, the presence of the target analyte is detected by impedance signal. In certain embodiments, the detectable signal comprises a change in impedance as a function of frequency. In certain embodiments, the presence of the target analyte is detected by amperometric or potentiometric signal.

In certain embodiments, the set of electrodes comprises a micro-interdigitated gold electrode. In certain embodiments, the set of electrodes consists of two electrodes. In certain embodiments, the set of electrodes comprises four electrodes. In certain embodiments, the detectable signal is displayed on the microprocessor through radio frequency identification (RFID). In certain embodiments, the biosensor is integrated into a medical, dental, or veterinary device having a tissue-contacting surface.

In certain embodiments, the target analyte comprises Staphylococcus aureus. In certain embodiments, the target analyte comprises methicillin-resistant Staphylococcus aureus (MRSA). In certain embodiments, the target analyte comprises Streptococcus pyogenes, Streptococcus pneumoniae, or Streptococcus agalactiae. In certain embodiments, the target analyte comprises a virus, or portion thereof. In certain embodiments, the target analyte comprises a molecule, or portion thereof, that is a marker for a cancer.

In certain embodiments, the SAM comprises mercaptoproprionic acid (MPA), 11-mercaptoundecanoic acid (MUA), 1-tetradecanethiol (TDT), or dithiobios-N-succinimidyl propionate (DTSP).

In another aspect, there is provided herein a biosensor which wirelessly communicates a readout to a display device.

In another aspect, there is provided herein a kit comprising the biosensor device described herein.

In another aspect, provided is a biosensor comprising a transducer component comprising a set of electrodes operatively connected to a microprocessor, the microprocessor being adapted to receive, process, and transmit a signal, where the set of electrodes includes at least one working electrode, and a receptor component having a sensing element capable of binding to at least one target analyte present in a sample, where the sensing element is attached to the working electrode either directly or via a self-assembled monolayer (SAM) layer positioned between and in contact with the sensing element and the working electrode, the transducer component and the receptor component being capable of being brought into direct contact with the sample in situ, where the sensing element, in the presence of a target analyte in a sample, causes a detectable signal capable of being transmitted to the working electrode, and where the biosensor wirelessly communicates a readout to a displaying device.

In another aspect, provided is a biosensor for detecting the presence of a target analyte, the biosensor comprising a removable chip comprising a set of electrodes formed thereon and a connector end, the set of electrodes including a working a electrode, where the connector end is configured to electrically connect the set of electrodes to a reader, a sensing element capable of binding to at least one target analyte, where the sensing element is attached to the working electrode either directly or via a self-assembled monolayer (SAM) layer positioned between and in contact with the sensing element and the working electrode, and a processing device configured to receive a signal from the set of electrodes when the chip is connected to the reader and display a graphical user interface, where the sensing element, in the presence of the target analyte, causes a detectable signal capable of being transmitted to the working electrode and received by the processing device. In certain embodiments, the biosensor includes an anti-fouling agent attached to the working electrode. In certain embodiments, the set of electrodes comprises the working electrode, a reference electrode, and a counter electrode.

In another aspect, provided herein is a chip for a biosensor device, the chip comprising a support defining an elongated surface and having a connector end and a testing area, a set of electrodes deposited on the testing area of the surface, where the set of electrodes includes a working electrode, and a sensing element linked to the working electrode either directly or via a SAM linker, where the sensing element is capable of binding to a target analyte, and where the sensing element, in the presence of a target analyte, causes a detectable signal capable of being transmitted to the working electrode. In certain embodiment, the chip further comprises an anti-fouling agent on the working electrode. In certain embodiments, the sensing element includes an antibody or other protein, nucleic acid, peptide, or aptamer.

In another aspect, there is provided herein a method of making a biosensor capable of detecting a target analyte in situ in a sample. The method generally includes linking a sensing element to a working electrode either directly or via a self-assembled monolayer (SAM); and operatively connecting a microprocessor to the working electrode such that, when the sensing element binds to a target analyte present in situ in a sample, the microprocessor detects and transmits a signal.

In another aspect, there is provided herein a method of detecting a bacterial infection in a living organism, which includes placing the biosensor device described herein at least partially in or on the living organism sufficient to come into contact with any bacterial target analyte present in the living organism; and, detecting the presence of the bacterial target analyte when the biosensor device transmits the detectable signal.

In certain embodiments, the biosensor device determines whether the bacterial target analyte is Gram-positive or Gram-negative, and the biosensor device transmits a signal to the medical instrument indicating whether the bacterial target analyte is Gram-positive or Gram-negative.

In certain embodiments, the change in the physical properties of the sensing matrix that is detected comprises the change in impedance as a function of frequency.

The biosensor may be adapted and incorporated into any of several suitable medical instruments or surgical tools, including on the flexible tip of an elongated medical instrument. In certain embodiments, the sensing element comprises antibodies, and the sensor is adapted to detect the presence of a bacteria.

### BRIEF DESCRIPTION OF THE DRAWINGS

The patent or application file may contain one or more drawings executed in color. Copies of this patent or patent application publication with color drawings will be provided by the Patent Office upon request and payment of the necessary fee.

FIG. 1A: Schematic representation of a biosensor device operatively connected at a distal end of a flexible tip of a medical instrument.

FIG. 1B: Schematic representation of another embodiment of an instrument that either incorporates a biosensor device and/or can be configured to have a biosensor device operatively attached to the instrument.

FIG. 2: Schematic representation of a portion of a biosensor device.

FIG. 3A: Schematic side elevational representation of an embodiment of an electrode useful in a biosensor device.

FIG. 3B: Cross-sectional schematic representation of an electrode having a working electrode, a counter electrode, and a reference electrode.

FIG. 3C: Perspective view of a protective membrane useful with the electrode shown in FIG. 3B.

FIG. 4: Schematic representation of a method for detecting a target analyte.

FIG. 5: Graph depicting a shifted sine wave current response to an applied sine wave voltage.

FIG. 6A: Schematic diagram of a circuit design for an electronic control system for use with the biosensor device illustrated in FIG. 1.

FIG. 6B: Box diagram of an electronic control system for use with the biosensor illustrated in FIG. 1 and FIG. 6A.

FIG. 7: Impedance curve showing an impedance shift when the surface of a bare gold electrode is modified by a SAM deposit.

FIG. 8: Potentiostatic electrochemical impedance spectroscopy (EIS) impedance curve showing a gold electrode with MPA SAM has a higher impedance magnitude and a different phase shift than a bare gold electrode.

FIG. 9: Cyclic voltammogram showing that a bare gold electrode has a higher maximum current, and therefore lower resistance, than a gold electrode with MPA SAM.

FIG. 10: Cyclic voltammogram showing a comparison between a bare gold electrode, a gold electrode with 3-MPA SAM, a gold electrode with 3-MPA and 11-MUA SAM, and a gold electrode with 11-MUA SAM. The curves show the gold electrode with 11-MUA SAM has the highest resistance.

FIG. 11: EIS impedance curves for four electrodes: a bare gold electrode, a gold electrode with 3-MPA SAM, a gold electrode with 3-MPA and 11-MUA SAM, and a gold electrode with 11-MUA SAM. The gold electrode with 11-MUA SAM was shown to have the highest impedance and the most distinct phase shift trend.

FIG. 12: Impedance curves generated by the sensing matrix comprising 11-MUA/MRSA antibody when exposed to serial dilutions of purified methicillin-resistant Staphyloccus aureus (MRSA) specific protein PBP2a in PBS for 10 minutes.

FIG. 13: Impedance curves generated by the sensing matrix comprising 11-MUA/MRSA antibody when exposed to 1 ng/ml of purified MRSA specific protein PBP2a in PBS for the time periods indicated.

FIG. 14: Impedance curve generated by the sensing matrix comprising 11-MUA/MRSA antibody when exposed to the culture of 10⁶ cells/ml MRSA, 10⁶ cells/ml non-resistant Staphylococcus aureus, or blank culture medium.

FIG. 15: Impedance changes when the sensing matrix comprising 11-MUA/MRSA antibody was exposed to a mixture of total 10⁶ cells/ml of MRSA and non-resistant Staphylococcus aureus. The shift of the curves corresponded to increased MRSA in the solution.

FIG. 16: Schematic illustration of a non-limiting embodiment of the biosensor system.

FIG. 17: Schematic illustration of a non-limiting embodiment of a signal processor circuit of the biosensor.

FIG. 18: EIS curves showing the sensitivity of the biosensor for MRSA. The slopes and magnitude (Z, ohm) change during sensing due to the increasing binding of bacteria. The concentrations of bacteria for the regular Staphylococcus solution and MRSA solution was 5×10⁹ cells/ml.

FIG. 19: Impedance curves at lower frequencies (100 mHz, for example) are lowered when the solution is diluted by 10 times. The concentration of bacterial was gradually diluted from 5×10⁹ to 5×10⁸ and even further to 5×10⁷.

FIG. 20: EIS curves for regular Staphylococcus.

FIG. 21: Impedance curves generated by a biosensor in the presence of Salmonella bacteria. Sensors for Salmonella bacteria were developed using anti-S. typhimurium LPS antibody (Abcam, Cambridge, Mass.) as the sensing element. Bacterial cultures were prepared by inoculating S. typhimurium (ATCC, Manassas, Va.) in nutrient broth, harvested after 16 hours, and diluted to 10⁵ CFU/ml. Fresh nutrient broth without bacteria served as control (blank) samples.

FIG. 22: Impedance curves generated by a biosensor in the presence of Listeria bacteria. Sensors for Listeria bacteria were developed using anti-Listeria antibody (RayBiotech, Norcross, Ga.) as the sensing element. Bacterial cultures were prepared by inoculating L. monocytogenes (ATCC, Manassas, Va.) in nutrient broth, harvested after 16 hours, and diluted to 10⁵ CFU/ml. Fresh nutrient broth without bacteria served as control (blank) samples.

FIGS. 23A-23B: An embodiment of a biosensor that allows individuals to do testing at home. FIG. 23A shows a chip containing an electrode with the sensing element thereon, and FIG. 23B shows a color photograph of a biosensor reader capable of reading the results from the chip.

FIGS. 24A-24B: Embodiments of a handheld biosensor, where the graphical user interface depicts a selection menu for particular infection types (FIG. 24A) and where the graphical user interface displays the raw impedance versus frequency curve generated from the sample (FIG. 24B).

FIGS. 25A-25B: An embodiment of a biosensor configured for use in an operating theater. FIG. 25A shows the entire assembly, and FIG. 25B is a close-up view of the chip (having the sensing element thereon) that is inserted into a reader connectable to the readout device.

### DETAILED DESCRIPTION OF THE INVENTION

It is to be understood that both the foregoing general description and the following detailed description are exemplary and explanatory only and are not intended to limit the scope of the current teachings. In this application, the use of the singular includes the plural unless specifically stated otherwise.

The use of the word “a” or “an” when used in conjunction with the term “comprising” in the claims and/or the specification may mean “one,” but it is also consistent with the meaning of “one or more,” “at least one,” and “one or more than one.” Also, the use of “comprise”, “contain”, and “include”, or modifications of those root words, for example but not limited to, “comprises”, “contained”, and “including”, are not intended to be limiting. The term “and/or” means that the terms before and after can be taken together or separately. For illustration purposes, but not as a limitation, “X and/or Y” can mean “X or Y” or “X and Y”. Throughout the entire specification, including the claims, the word “comprise” and variations of the word, such as “comprising” and “comprises” as well as “have,” “having,” “includes,” and “including,” and variations thereof, means that the named steps, elements, or materials to which it refers are essential, but other steps, elements, or materials may be added and still form a construct within the scope of the claim or disclosure. When recited in describing the invention and in a claim, it means that the invention and what is claimed is considered to be what follows and potentially more. These terms, particularly when applied to claims, are inclusive or open-ended and do not exclude additional, unrecited elements or method steps.

Various embodiments are described herein in the context of apparatus, method, system, and/or process for sensing target analytes, such as bacteria or viruses or portions thereof. Those of ordinary skill in the art will realize that the following detailed description of the embodiments is illustrative only and not intended to be in any way limiting. Other embodiments will readily suggest themselves to such skilled persons having the benefit of this disclosure. Reference to an “embodiment,” “aspect,” or “example” herein indicate that the embodiments of the invention so described may include a particular feature, structure, or characteristic, but not every embodiment necessarily includes the particular feature, structure, or characteristic. Further, repeated use of the phrase “in one embodiment” does not necessarily refer to the same embodiment, although it may.

In the interest of clarity, not all of the routine features of the implementations or processes described herein are shown and described. It will be appreciated that numerous implementation-specific adaptations are incorporated to achieve specific goals, such as compliance with application- and business-related constraints, and that these specific goals vary from one implementation to another and from one developer to another. Moreover, it will be appreciated that such a development effort might be complex and time-consuming, but would nevertheless be a routine undertaking for those of ordinary skill in the art having the benefit of this disclosure.

Described herein is a biosensor device for detecting the presence and/or determining the amount of a target analyte in situ and in real time. The biosensor provides a simple and easy-to-handle device capable of detecting the presence and/or concentration of any kind of bacteria on a sample. In certain embodiments, the biosensor device can calibrated to both detect and quantify an amount of a target analyte present.

Generally, the biosensor includes an electrode set that is composed of a working electrode, a reference electrode, and a counter or auxiliary electrode. In general, a working electrode is an electrode being studied. A counter or auxiliary electrode is an electrode that completes the current path. A reference electrode is an electrode that serves as an experimental reference point. For clarity, reference to an “electrode” herein, without further limitation, refers to a working electrode. The working electrode of a biosensor described herein has biomolecular probes attached that can generate an electrochemical reaction which results in a signal. Processing this signal can release more information about the electrochemical reaction. Any sample of, for instance, blood, saliva, sweat, or breath is provided to the electrode of the device, and the biosensor analyzes a change in physical property, such as the impedance change of the electrochemical reaction on different low frequencies. The degree of change in the physical property (e.g., impedance) is proportional to the amount of target analyte present in the sample. Thus, the amount of target analyte can be quantified.

In some embodiments, the whole signal processing system is provided by a battery, without using AC power supply. This approach not only makes the device handheld and portable, but also reduces a lot of power consumption. The biosensor's measurement is controlled by a mixed-signal microprocessor, and the measured data can be sent to a smart phone, tablet, or LCD display (e.g., via Bluetooth technology) or any displaying system for storing and showing the graphs. That is, a readout can be wirelessly communicated to a displaying system. FIG. 17 shows a non-limiting example of a circuit diagram for the biosensor. In some embodiments, the biosensor includes an ARM architecture based microcontroller having multitasking capabilities, a current-to-volage amplifier to measure current digitally, and non-inverting amplifier, a battery, and a displaying device, such as an LCD or a smart phone.

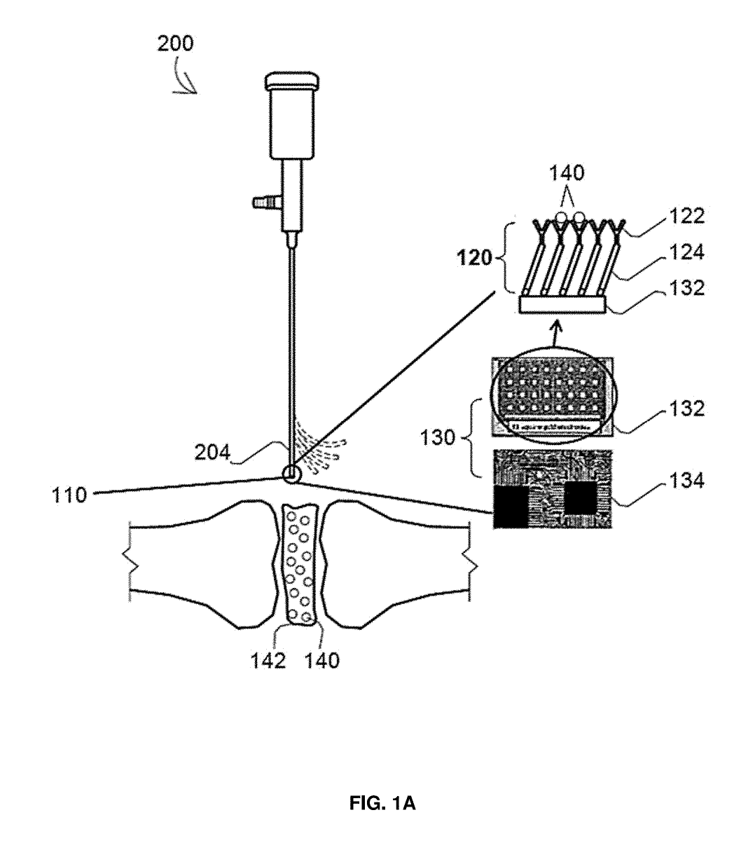

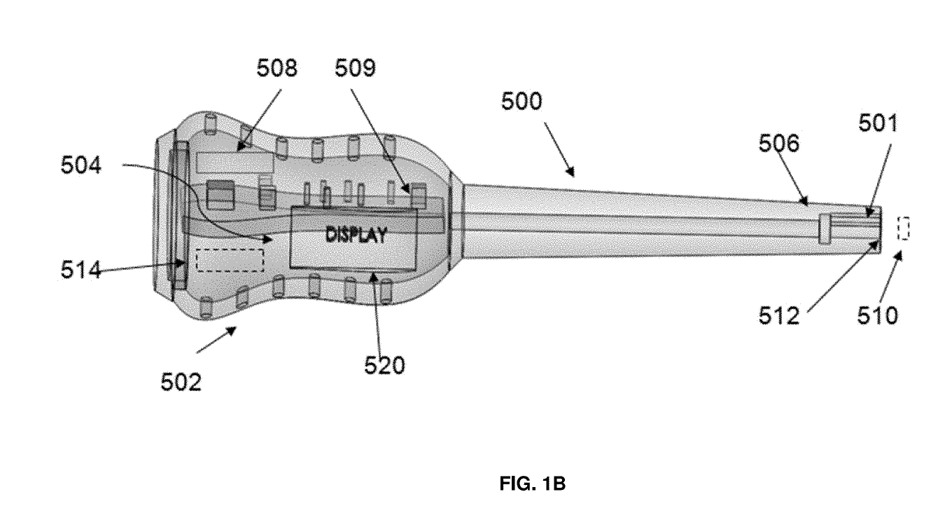

Referring first to a schematic representation of one embodiment of a biosensor device shown in FIGS. 1-3, a biosensor device 110 generally includes a receptor component 120 and a transducer component 130. The receptor component 120 includes at least one sensing element 122, and may include a self-assembled monolayer (SAM) layer 124. However, as will be discussed in more detail below, the SAM layer 124 is not necessary and some embodiments of the receptor component 120 do not include a SAM layer 124, although the presence of a SAM layer 124 has been found to be beneficial for low frequency measurements. The transducer component 130 is responsive to changes that occur in the receptor component 120 from the interaction between a sensing element and a target analyte for generating measurable signals, as further explained herein.

Referring now to FIG. 1A, an example of a biosensor 110 that is operatively connected to an instrument 200 is shown. For ease of explanation, the biosensor device 110 is shown as being positioned on a contact member 204 such as a distal end of a flexible tip of the instrument 200. It is to be understood, however, that other embodiments are within the contemplated scope of the disclosure herein.

The transducer component 130 is composed of a set of electrodes 132, and at least one microprocessor 134. The microprocessor 134 is adapted to transmit and process a signal, as further explained herein. It is to be understood that the function of the microprocessor 134 can include the generating of an electronic image for review by a skilled person. Further, it is understood that the microprocessor 134 can be replaced by any suitable processing device. Also, in certain embodiments, the biosensor device 110 can include more than one transducer component 130. In such embodiments, each electrode 132 can be operatively connected to a corresponding microprocessor 134. As will be discussed in more detail, some embodiments of the biosensor involve the electrodes 132 being on a separate element (such as a removable chip) from a processing device.

As mentioned above, the receptor component 120 includes one sensing (or receptor) element 122 and, optionally, a self-assembled monolayer (SAM) 124. The sensing element 122 is capable of binding to at least one target analyte 140. The self-assembled monolayer (SAM) 124, when present, is positioned between, and is in contact with, both the sensing element 122 and a working electrode 152 of the electrode set 132. The sensing element 122, in the presence of the target analyte 140, causes a detectable signal capable of being transmitted to the working electrode 152. When the sensing element 122 contacts a sample 142 (for example, a fluid or tissue), and binds to the target analyte 140 that is present in the sample 142, a change in at least one physical property is detected by the set of electrodes 132, and can be transmitted as a signal by the microprocessor 134. As further described herein, in certain embodiments, the change in the physical property that is detected comprises the change in impedance as a function of frequency.

FIG. 1B is a schematic representation of another embodiment of an instrument 500 that either incorporates a biosensor device 501 and/or can be configured to have a biosensor device 510 operatively attached to the instrument 500. The instrument 500 generally includes a proximal handle end 502 that defines an annular opening 504, and a distal probe end 506 that is configured to hold either the integral biosensor 501 or the attachable biosensor device 510. It is to be understood that the biosensor devices 501/510 can generally include a transducer component (e.g., electrode and microprocessor) and a receptor component (e.g., SAM and sensing element) that are similarly configured as described elsewhere herein. It is also to be understood that such electrodes are positioned to come into contact with a power source 508, such as a battery, and a microprocessor. In some embodiments, the biosensor 501/510 can be attached to the distal probe end 506 of the handle 502 in a suitable manner. For example, the distal probe end 506 can define one or more detents 512 so that the biosensor 501 can be snapped onto the distal probe end 506. In another embodiment, the biosensor 501 can be screwed or threaded onto the distal probe end 506. In certain embodiments the biosensor device 501/510 can be either removably attached, or permanently attached, to the distal probe end 506.

In certain embodiments, at least one of the entire instrument 500, the biosensor device 501/510, the handle end 502, and/or the distal probe end 506 can be disposable and/or configured to be attached and used in a sterile condition.

In certain embodiments, the annular opening 504 can also be configured to contain an RFID 514 for transmitting the detected signal. In addition, the instrument 500 can include a display 520 that is operatively connected to the microprocessor. The display 520 can be configured to display different types of information; for example, “+” or “−”, type of target analyte present, quantitative amount of analyte present, and the like.

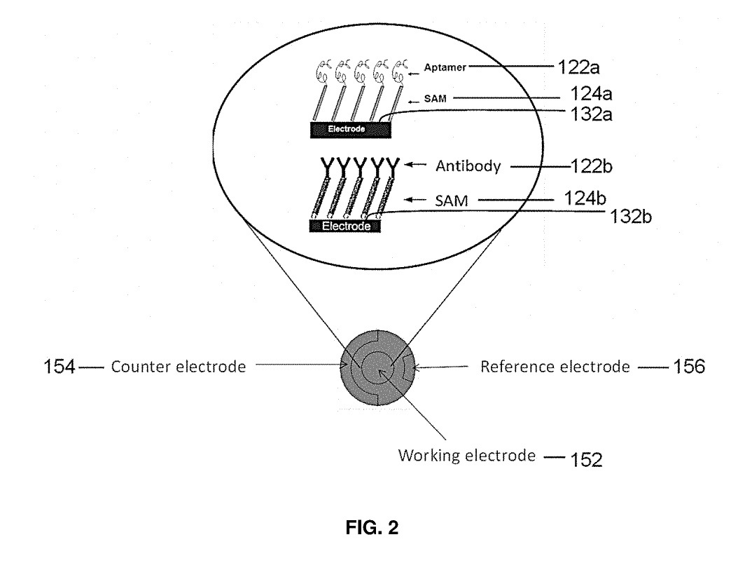

In certain embodiments, as schematically illustrated in FIG. 2, multiple target analytes can be sensed simultaneously. For example, the biosensor device 110 can include an electrode set 132 having a first side 110a on which a first SAM layer 124a is affixed, and a second side 101b on which a second SAM layer 124b is affixed. The first SAM layer 124a can be attached to a first sensing element 122a, and the second SAM layer 124b can be attached to a second sensing element 122b, where the first and second sensing elements 122a, 122b are selective for different analytes.

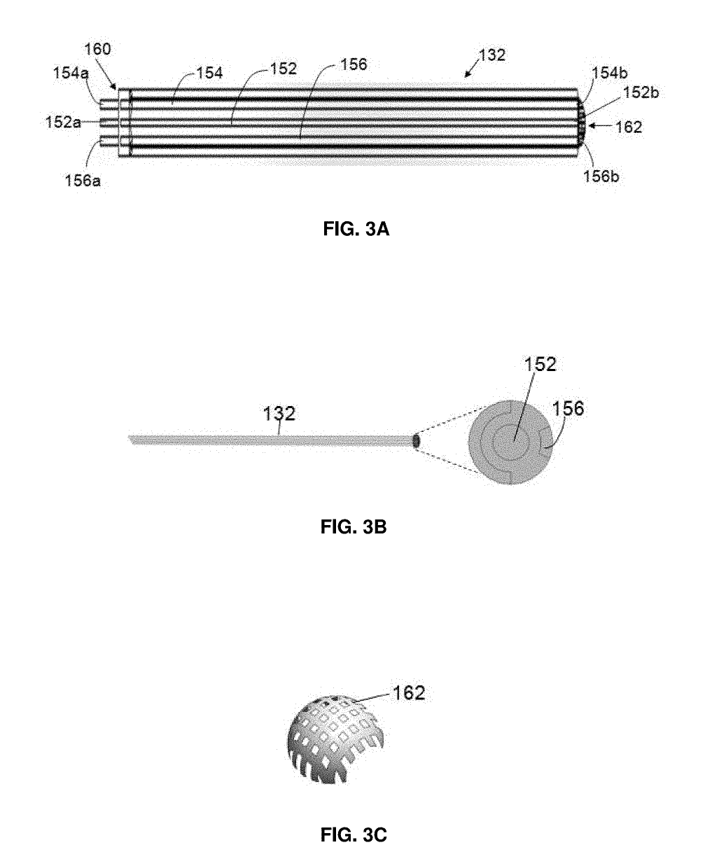

FIGS. 3A-3C show one embodiment of an electrode set 132 suitable for use in the biosensor device 110. The electrodes 132 can include a working electrode component 152, a counter electrode component 154, and a reference electrode component 156. It is understood, however, that the electrodes 132 can include more or fewer electrodes, such as a set of two electrodes or a set of four electrodes. A set of two electrodes generally includes a working electrode 152 and a reference electrode 156, while a set of four electrodes generally includes an additional working sense electrode. In the embodiment shown, the working electrode component 152, the counter electrode component 154, and the reference electrode 156 each have proximal ends 152a, 154a, 156b that are integrated on a first end 160 of the electrode 132. The first end 160 can be configured to be connected to a socket of an impedance analyzer. In certain embodiments, one or more of distal ends 152b, 154b, 156b of the working electrode component 152, the counter electrode component 154, and the reference electrode component 156, can be protected by a suitable membrane 162. In the embodiments shown in FIG. 3C, the membrane 162 comprises an electrode mesh.

Referring back to FIG. 2, there is shown a pathogen-specific aptamer 122a linked to the first side 110a of the electrode set 132 via a first SAM layer 124a. Also shown is a pathogen-specific antibody 124b linked to a second side 110b (or, as alternately shown as a second electrode 110b) of the working electrode 152 via a second SAM layer 124b.

In certain embodiments, the three electrode system (working 152, counter 154, and reference 156 electrodes) are useful for the electrochemistry analysis of a reaction causing electrical current flow. The binding reaction occurs on the working electrode 152. The counter electrode 154 and the reference electrode 156 generate electrical potentials against other potentials to be measured.

The biosensor device can be configured to compensate for any noise at the time of the sampling where post-processing can include an algorithm that is applied through a software program to remove random noise, slopes, and the like. Furthermore, various parameters can be optimized experimentally, and such optimization is encompassed wiin the present disclosure. These parameters include, but are not limited to, electrode size, drive voltage, environmental conditions such as temperature, analyte binding concentration, and the like.

The biosensor device can be configured to be adapted for use on small (e.g., nanoscale) samples. Also, the receptor component 120 can be configured to have different sensing elements 122 that can be clustered or arrayed for use in detection of multiple target analytes 140.

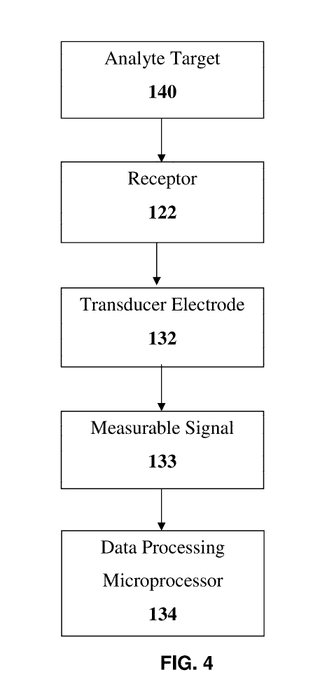

FIG. 4 depicts an example process flow diagram for using the biosensor device 110. When an analyte target 140 is present, there is a binding between the target analyte 140 and a target-specific receptor 122, which is, in turn, bound to the set of electrodes 132. The electrodes 132 detect a signal 133 (e.g., alteration in impedance, etc.) and a measurable signal 133 is generated. The measurable signal 133 is processed by the microprocessor 134, thereby detecting the presence or absence of the target analyte 140.

In general, the sensing element 122 can include any binding protein, peptide, nucleic acid, or polymer which produces an electrical impulse, such as a change in impedance, upon binding. By way of non-limiting examples, the sensing element 122 can include antibodies, enzymes, antibody fragments, DNA, RNA, aptamers, oligonucleotides, synthetic or natural polymers, or combinations or portions thereof. Suitable polymers include, but are not limited to, alginate, chitosan, or carboxymethyl cellulose (CMC), and derivatives thereof. The terms “antibody” and “antibodies” as used herein refer to proteins used by the immune system to identify and/or neutralize foreign targets such as bacteria or viruses. Antibodies tend to be Y-shaped glycoproteins produced by B-cells and secreted by plasma cells. Antibodies recognize particular parts of a target known as antigens and bind to a specific epitope thereon. “Antibody” can be used interchangeably with “immunoglobulin” and is meant to include all known isotypes and natural antibodies.

It is understood that the identity of the sensing element will depend on the type of target analyte desired to be detected. For example, when heavy metals are to be detected, the sensing element 122 may include a polymer such as alginate as the receptor. Because heavy metals are positively charged, a negatively charged polymer such as alginate interacts with the heavy metals, and this interaction causes a detectable change in impedance on the electrode. As another example, when a bacteria or virus is the target analyte to be detected, the sensing element 122 may include one or more antibodies or antibody fragments. In certain embodiments, the sensing element comprises antibodies specific for a target analyte to be sensed, such as Staphylococcus aureus antibodies in a sensor designed to detect the presence of Staphylococcus aureus. The antibodies can be synthesized or bought commercially.

An electrode generally refers to a composition, which, when connected to an electronic device, is able to sense a current or charge and convert it to a signal. Alternatively, an electrode can be a composition which can apply a potential to, and/or pass electrons to or from, connected devices. Different electrode materials include, but are not limited to, certain metals and their oxides, including gold; platinum; palladium; silicon; aluminum; metal oxide electrodes including platinum oxide, titanium oxide, tin oxide, indium tin oxide, palladium oxide, silicon oxide, aluminum oxide, molybdenum oxide (Mo₂O₆), tungsten oxide (WO₃) and ruthenium oxides; and carbon (including glassy carbon electrodes, graphite, and carbon paste). In one embodiment, the electrode can be a micro interdigitated gold electrode (MIGE). The membrane 162, or electrode mesh, may be composed of any suitable material that does not interfere with the interactions between the analyte and the sensing element 122. Non-limiting examples of suitable materials for the membrane 162 include polymers, plastics, fibers, low conductivity metals, and ceramics. In one embodiment, the membrane 162 is composed of synthetic fibers.

The SAM layer 124, when present, generally comprises a surface deposit on a surface of the electrode set 132, or at least on the surface of the working electrode 152, and may include any suitable linking molecules between the sensing element 122 and the electrode 132. Depending on the target analyte 140 to be detected, the SAM layer 140 can substantially cover, or can partially cover, an area on the surface of the electrode set 132 or working electrode 152. The SAM layer 124 generally comprises one or more organic molecules such that the SAM molecules act as a linker between the sensing element 122 and the electrode 132. The SAM layer 124 can be modified with a suitable functional group, such as an amino group, in order to better facilitate binding of various receptor types (such as a nucleic acid or a polymer) to the SAM layer 124. As one non-limiting example, a SAM is formed with mercaptoproprionic acid (MPA), which is readily bound with the amino group in certain antibodies via covalent bonding. In other non-limiting embodiments, a SAM is made from 11-mercaptoundecanoic acid (MUA), 1-tetradecanethiol (TDT), or dithiobios-N-succinimidyl propionate (DTSP). One suitable method of making and characterizing a monolayer is described in chapter 6 of Electrochemistry—A Laboratory Textbook; A workbook for the 910 PSTAT mini, Barbara Zumbrägel, Metrohm Monograph, January, 2013, the disclosure of which is hereby incorporated by reference. As additional non-limiting examples, the SAM layer 124 may be composed of a gel, a polysaccharide, a polymer, or combinations thereof.

Although the presence of a SAM layer 124 is beneficial for impedance measurements at low frequencies, a SAM layer 124 is not necessary. Instead, the sensing element 122 may be attached directly to the electrode set 132, or directly to at least the working electrode 152. This can be accomplished by modifying the sensing element 122 with a functional group, such as a thiol or phosphate group, to facilitate binding directly to the electrode. Thus, the sensing element 122 may be attached to the working electrode directly, via a SAM layer 124, or through some combination of both.

In some embodiments, one or more anti-fouling agents that can repel other proteins are incorporated into the SAM layer 124 (if present), for instance in the form of a binary (i.e., consisting of two mono-layers) SAM layer, or otherwise attached to the set of electrode 132 or working electrode 152. Anti-fouling agents function to get proteins other than the proteins (or other molecules) being detected away from the sensing molecules (e.g., antibodies) of the sensing matrix. Thus, a binary SAM layer can include one SAM that optimizes stability (i.e., attachment) of the antibodies, and one SAM that prevents the adhesion of non-specific proteins while maintaining the stability of attached antibodies (i.e., provides anti-fouling). The anti-fouling agents can be polymers, or any other suitable agents which repel non-specific proteins. For instance, a linear alkanedithiol can be used as an anti-fouling agent. In one non-limiting example, the anti-fouling agent is 3,6-dioxa-8-mercaptooctan-1-ol (DMOL). In another non-limiting example, the anti-fouling agent is (1-mercapto-11-undecyl)tri(ethylene glycol) (PEG3). PEG3 is resistant to protein adsorption, and so can be employed to prevent non-specific adsorption of proteins. In one non-limiting example of a method of making a biosensor with an anti-fouling agent, a binary self-assembled monolayer is made using DMOL and 11-MUA, and antibodies are then attached to the 11-MUA.

A binary SAM can be made, for example, by a reductive desorption process. For example, a SAM component having a low redox potential, such as 3-mercaptopropionic acid (MPA), can be adsorbed onto a gold or other metal surface along with PEG3 in an ethanol solution. The low redox potential means that the SAM can be easily eliminated by reductive desorption while leaving the PEG3 intact. The MPA can be desorbed from the metal electrode by applying an electric potential in a suitable solvent such as KOH. Then, the metal surface can be immersed in a solution of, e.g., 11-MUA to form 11-MUA layers on the metal surface that already contains PEG3 layers attached thereto, thereby producing a binary SAM composed of DMOL and PEG3 on the metal surface.

Various ratios of the two SAMs in a binary SAM layer can be used to create the binary SAM layer. Using 11-MUA and DMOL as examples, a binary SAM layer can be made of a 80:20, 50:50, or 20:80 ratio of 11-MUA to DMOL. In general, as the amount of DMOL increases, the anti-fouling effect against nonspecific proteins is enhanced, though the signal intensity may decrease. A 50:50 ratio of 11-MUA to DMOL produces adequate repelling of non-specific proteins and still generates a sufficient signal for target molecules. As shown in the examples herein, the use of the anti-fouling agent DMOL results in very good bacteria detection.

In one embodiment, the biosensor device detects electrochemical signals that may comprise, for example, conductivity signals, capacitance signals, impedance signals, potentiometric signals, or voltammetric signals. In embodiments comprising potentiometric sensors, a potential signal developed at the electrode/electrolyte surface is used to quantify the concentration of analyte present. In embodiments comprising voltammetric or amperometric sensors, a constant voltage signal is applied to the system and corresponding electrical current is used to quantify the analyte. Variable (linear or cyclic) voltage can be applied and the height of the peak in the current—voltage curve is used to quantify the analyte.

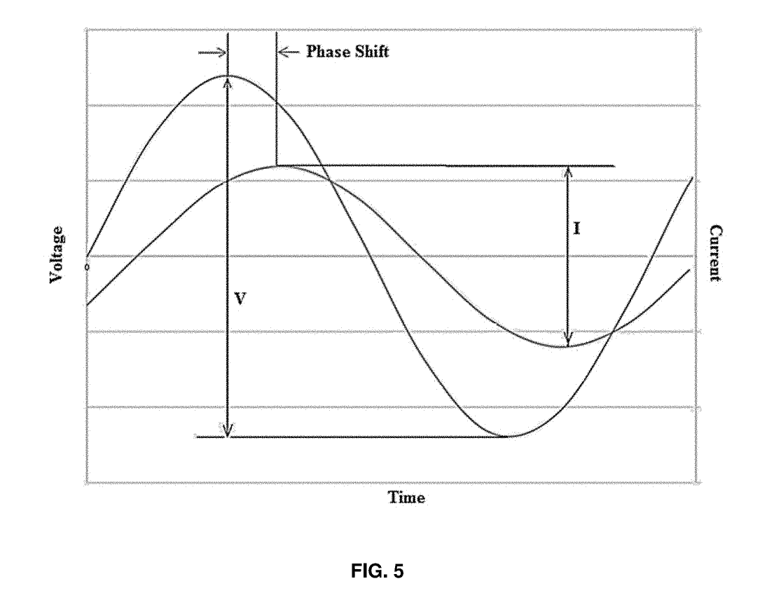

In some embodiments, the biosensor device utilizes electrochemical impedance spectroscopy, which measures impedance over a range of frequencies, to quantify the analyte. When a sine wave voltage is applied to a system, it produces a shifted sine wave current response. The impedance (Z) has two components: magnitude and phase shift (angle). This is illustrated in FIG. 5. The rate and degree of impedance change represent the presence and concentration of bacteria. Impedance can be calculated according to the equations:

$$\vert Z \vert = \frac{V}{I}$$

$$∅ = Phase shift$$

$$Z = \vert Z \vert {e}^{i ∅} ( polar coordinates )$$

$$Z = {Z}_{real} + i {Z}_{img} ( cartesian coordinates )$$

The microprocessor processes the signals and eventually displays the information. Signal processing can generally include a series of microelectronic channels that screen the sensor signals and control the noise, calibration, and amplification.

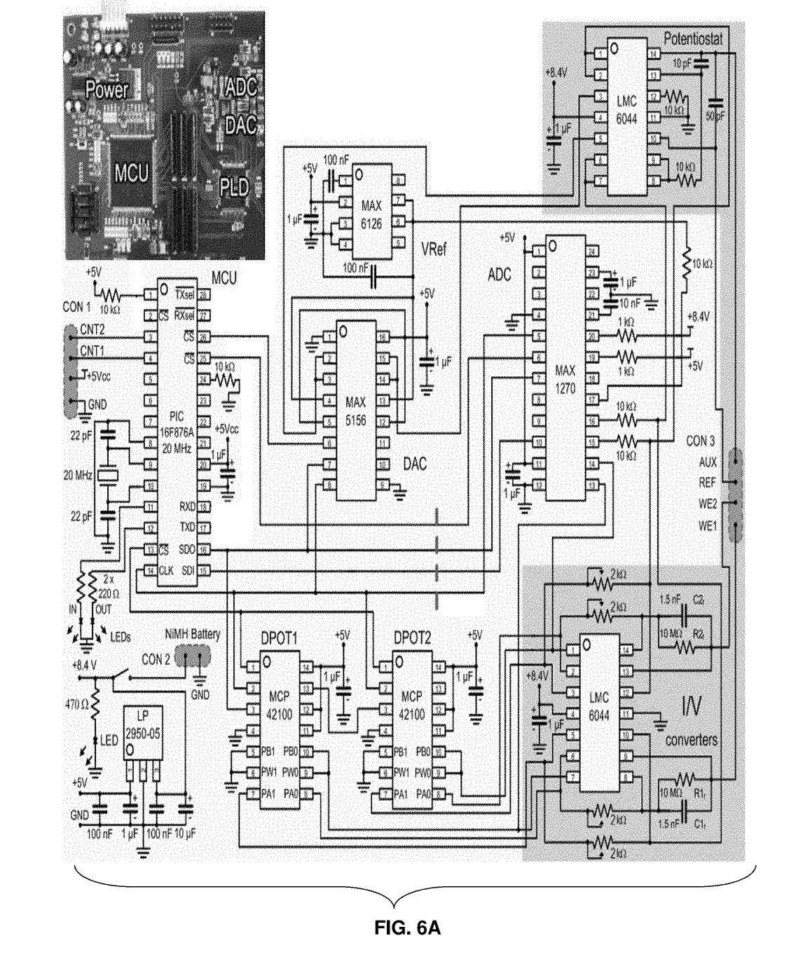

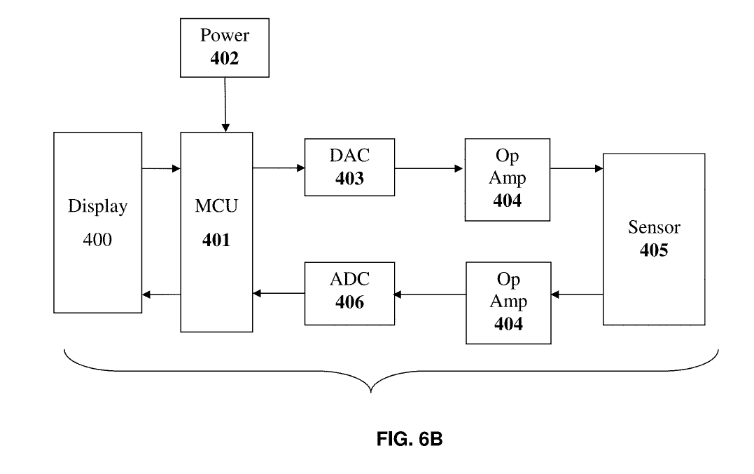

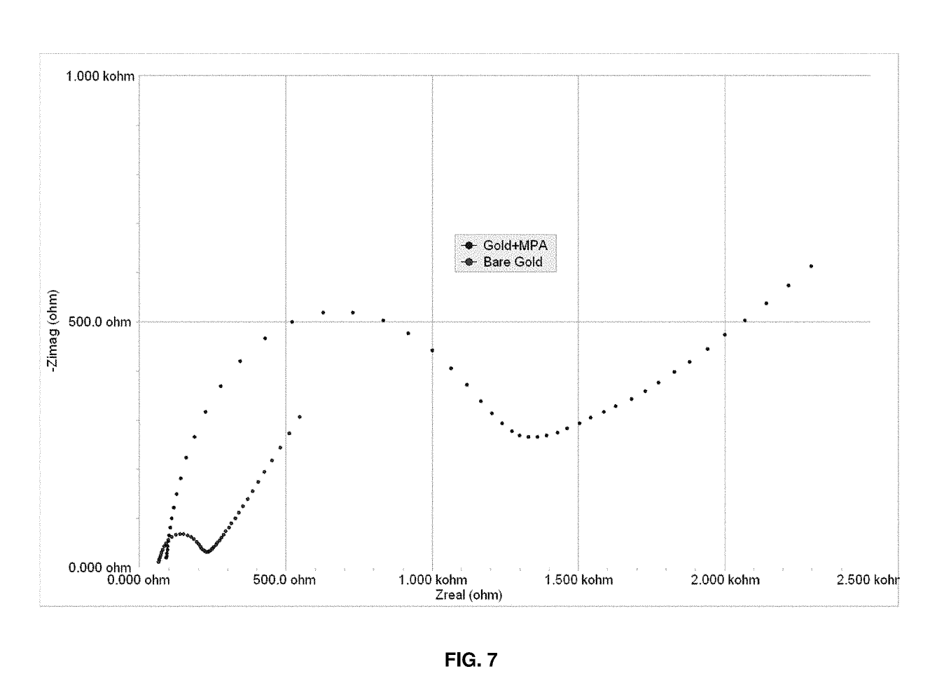

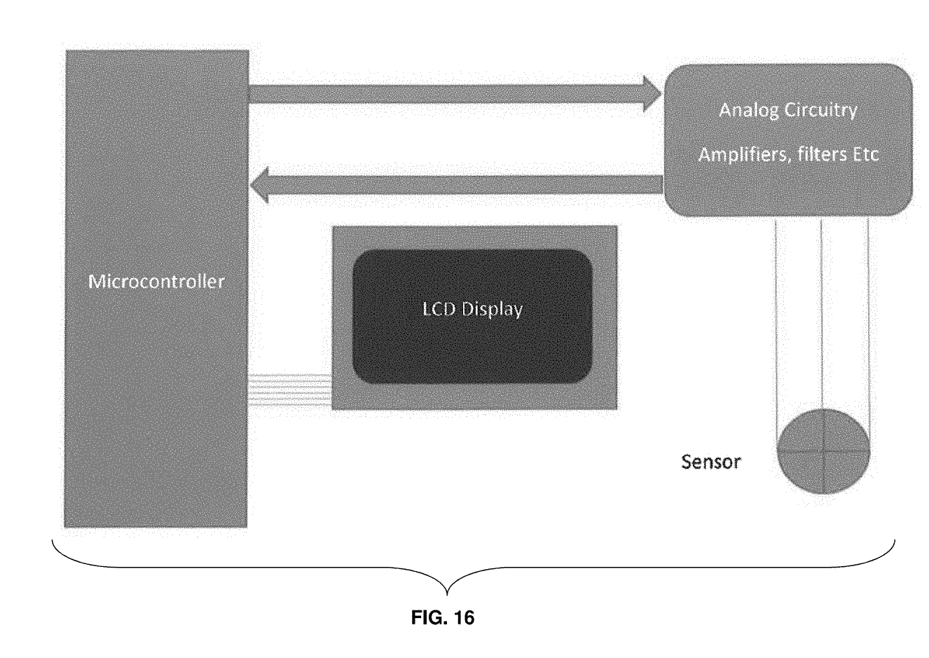

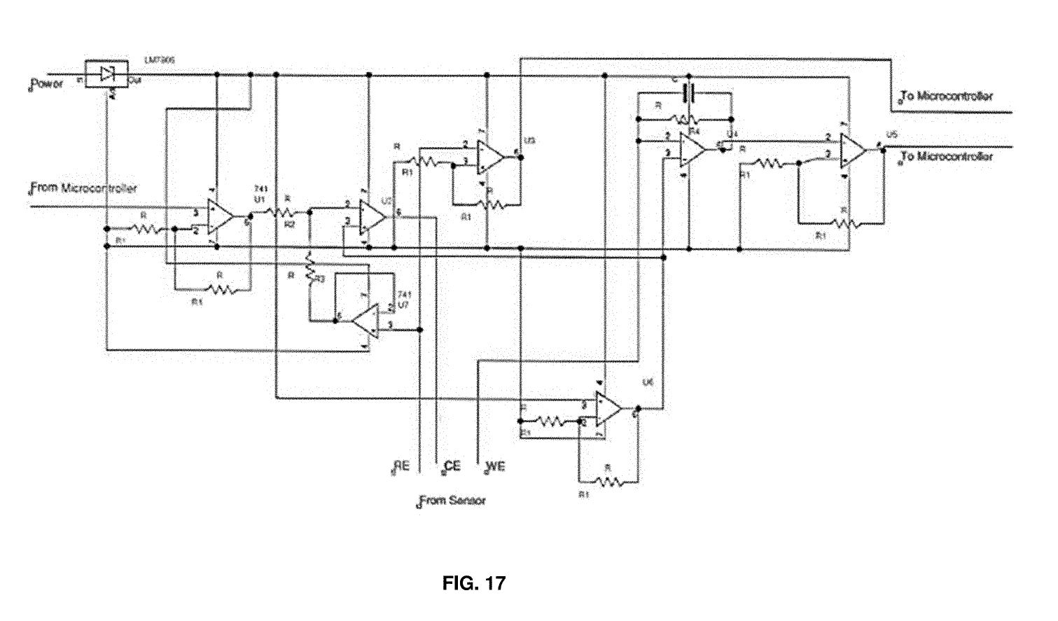

FIG. 6A is a schematic diagram of an exemplary electronic control system for use with the biosensor described herein. FIG. 6B is an exemplary flow diagram where an analog signal is generated in the form of a current, which is then amplified by an operational amplifier (Op Amp) 404 to reduce noises in the voltage applied to the electrode and the measured current signal, to switch current and voltage, and to control amplification. The amplified signal is then converted to a digital signal by an analog-to-digital converter (ADC) 406. The digital signal is controlled and processed by a micro-controller unit (MCU) 401, having a power supply 402, to produce a display 400. The micro-controller unit 401 can utilize specialized software programs to perform various functions. The controlled signal is processed through a digital-to-analog converter (DAC) 403 and converted to an analog signal. Conversion to an analog signal gives potential to the sensor 405; the analog signal becomes an additional potential to the electrode. In some embodiments, radio frequency identification (RFID) can be utilized to directly display the sensing information on a computer. FIG. 16 is a schematic illustration of a non-limiting example of the whole system.

In certain embodiments, the microprocessor is programmed to screen noise and to pick up impedance change at a very low frequency range (for example, from about 1 Hz to about 10 Hz). The microprocessor includes an algorithm program capable of screening background noise and detecting an up impedance signal that represents the presence and concentration of target analyte. Also, in certain embodiments, the detectable signal can be displayed on the microprocessor through radio frequency identification (RFID).

One embodiment of the circuit design for the biosensor is shown in FIG. 17. In this embodiment, a microcontroller is programmed to generate an analog wave form (such as a sine wave) using its own built-in Digital-to-Analog Converter (DAC). The output of this DAC is fed to a filter which smoothens the wave form and an amplifier (U1) which attenuates the waveform to the desired level. The frequency of the wave is controlled digitally through the program. The analog wave is then given to the inverting input of OpAmp (U2) and passed to the counter electrode (CE). The counter electrode completes the cell circuit and maintains the potential of the cell. The reference electrode is used to measure the potential of the cell and provide feedback (U7) to the counter electrode circuit. The voltage from the reference electrode is amplified (U3) and sent back to the microcontroller for processing. All the current from the electrochemical reaction is pushed to the working electrode, which is connected to the current-to-voltage converter (U4). The output of the current-to-voltage converter is then amplified (U5) and sent back to the microcontroller. Using this current value from the working electrode and the voltage from reference electrode, the microcontroller does the required processing to calculate the impedance of the sensor and display/store on the displaying device(s). When using a 5V system, a reference voltage is needed for opamp circuits to work. The opamp U6 is used to generate this reference voltage.

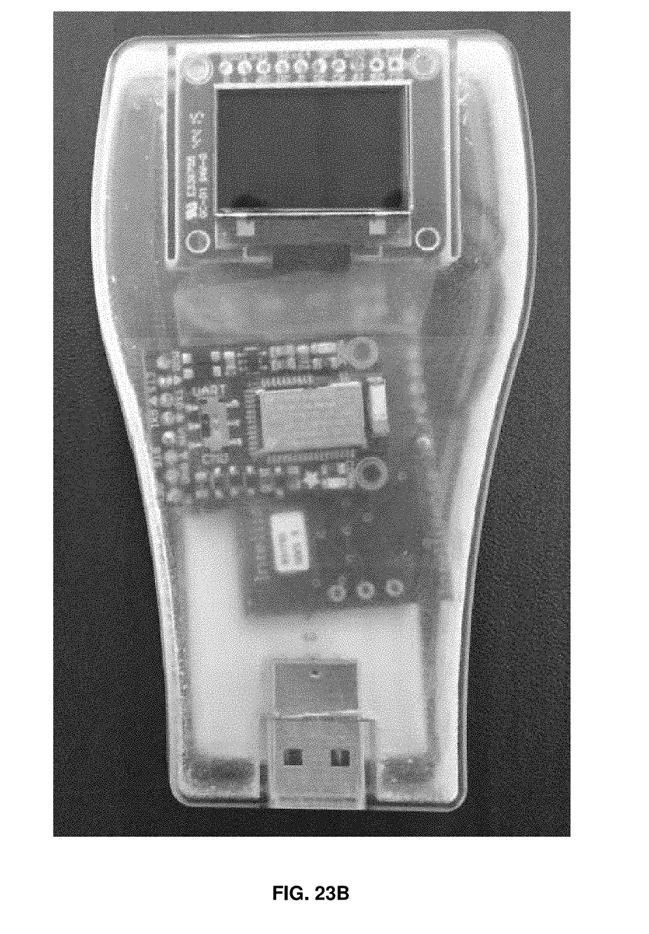

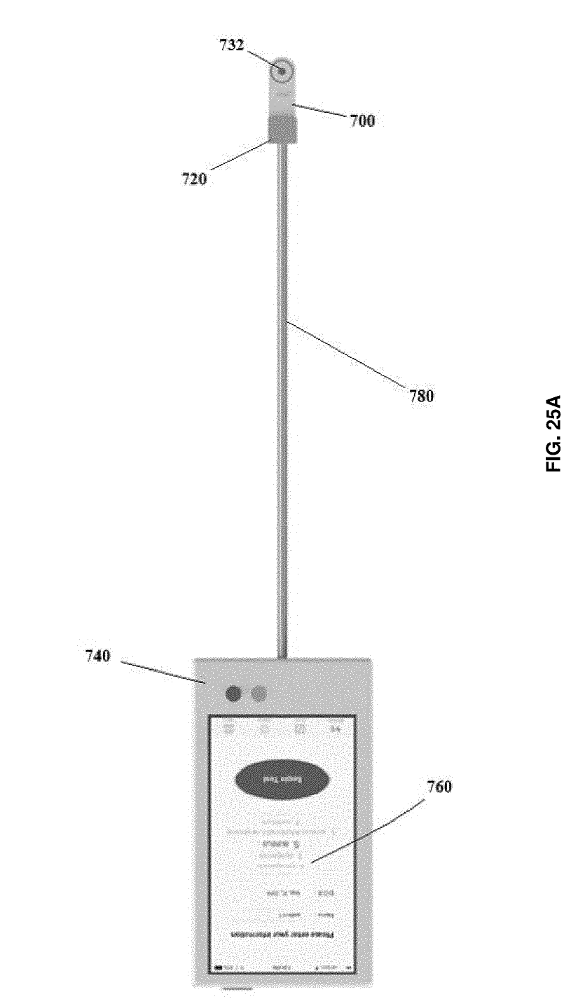

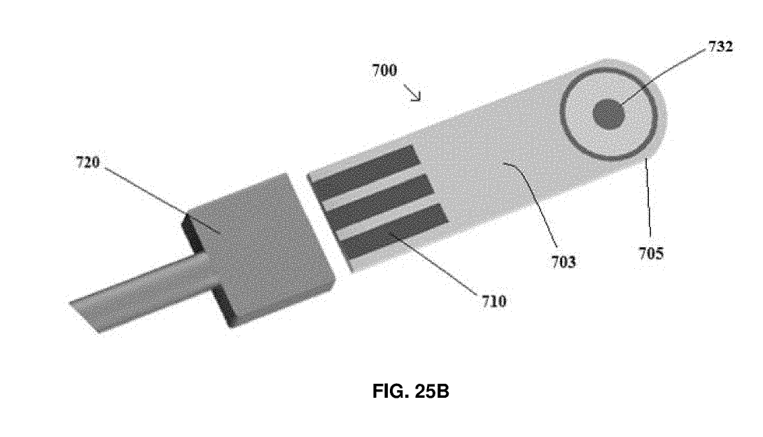

FIGS. 23-25 show alternative embodiments of the biosensor. As seen in FIG. 23A and FIG. 25B, the biosensor can include a removable chip 700 that contains the receptor component 120, and a separate readout or processing device 740. The removable chip 700 has a set of electrodes 732 printed or formed thereon, such as by a metal coating. The electrodes 732 can be a set of electrodes, such as depicted in FIG. 2, composed of a working electrode 152, a reference electrode 156, and a counter or auxiliary electrode 154. Alternatively, the set of electrodes 732 may be composed of only a working electrode and a reference electrode, or, as another option, may be composed of four electrodes: a working electrode, a working sense electrode, a reference electrode, and a counter or auxiliary electrode. In embodiments having four electrodes, two electrodes (the working and counter electrodes) can carry the current, and two electrodes (the working sense and reference electrodes) can be sense leads which can measure voltage.

The chip 700 can be fabricated from any suitable material to act as a support for the electrode set 732, including but not limited to glass, ceramic, polymers, plastics, silica, or combinations thereof. The removable chip 700 defines an elongated surface 703 of any suitable shape or design, the surface having a testing area 705 and a connector end 710, where the receptor component 120 is disposed on the testing area 705 and the connector end 710 is configured to electrically connect the electrode 732 to a reader 720. All of the electrodes in the set of electrodes 732 (namely, the working electrode component 152, the counter electrode component 154, and the reference electrode component 156, as illustrated in FIG. 2) can be electrically connected to a reader 720 by the connector end 710. The receptor component 120 on the chip 700 includes a sensing element 122 attached to the electrodes 732, or at least the working electrode in the set of electrodes 732, either directly or via a SAM layer 124. When present, the SAM layer 124 is positioned between, and in contact with, the sensing element 122 and at least one of the set of electrodes 732 (namely, the working electrode). Alternatively, as described above, the sensing element 122 can be attached to the working electrode of the electrode set 732 through a combination of direct attachment to the electrodes (or at least working electrode) and attachment via a SAM layer 124. The sensing element 122 includes any binding protein, peptide, nucleic acid, or polymer which produces an electrical impulse, such as a change in impedance, upon binding. The sensing element 122 is capable of binding to a target analyte and thereby causing a detectable signal transmitted to the working electrode. Optionally, an anti-fouling agent can be incorporated on the set of electrodes 732, or at least on the working electrode of the electrode set 732.

A processing device 740, such as a smart phone or tablet, is configured to receive the signal from the set of electrodes 732 via the reader 720, when the removable chip 700 is electrically connected to the reader 720. The processing device 740 reads the signal and generates a graphical user interface 760. Thus, the chip 700 generally does not include a microprocessor; rather, the signal is processed by the processing device 740. However, it is possible to incorporate a small microprocessor onto the chip 700, and such configurations are entirely encompassed within the present disclosure.

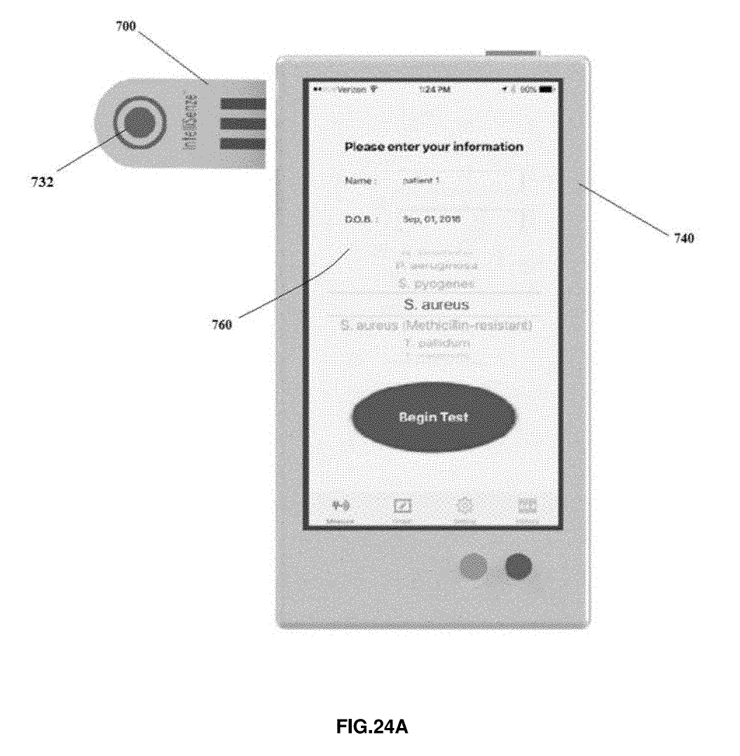

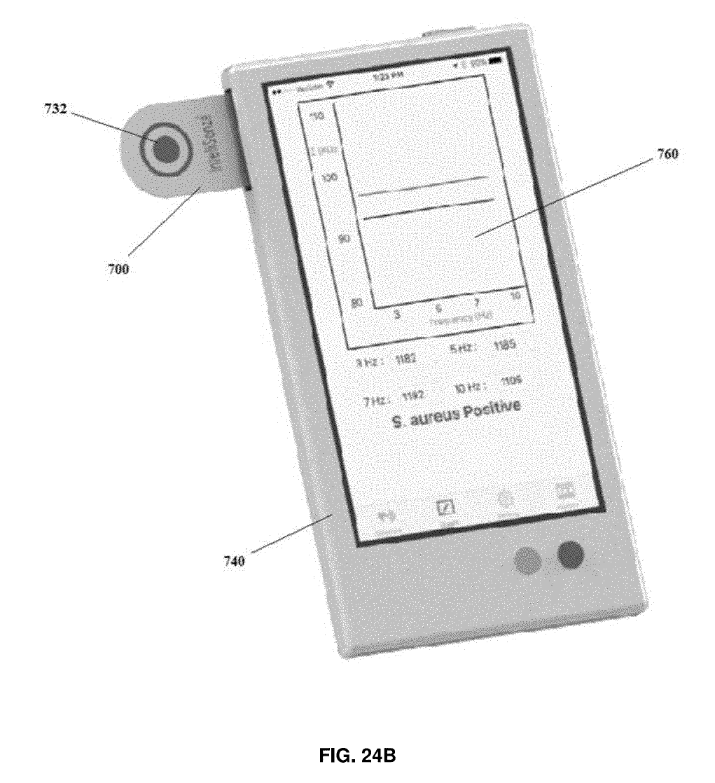

The testing area 705 of the chip 700 can be exposed to a sample, such as by dropping a liquid sample onto the set of electrodes 732, by immersing the target area 705 of the chip 700 into a liquid medium containing the target analyte, by inserting the chip 700 into or onto an anatomical location where a pathogen or toxin is to be detected, or any other method that is practical given the particular target analyte and sample type/environment. The chip 700 is insertable into a reader 720 before, during, or after exposure to the target analyte. The reader 720 facilitates the processing of the detectable signal by a suitable processing device 740, such as a smart phone, tablet, or computer via a suitable connection such as a USB or wireless (e.g., Bluetooth) connection. In some embodiments, the reader 720 can be connected to the processing device 740 by a suitable cable 780 of customizable length. Alternatively, the chip can be inserted into a handheld readout device, such as that seen in the photograph in FIG. 23B or the illustration in FIG. 24B. Thus, the reader 720 and the processing device 740 may actually be a single device. In any event, the processing device 740 acts to process the signal from the electrodes 732 on the chip 700, and generates a graphical user interface 760 displaying the results of the test.

The graphical user interface 760 is customizable. As seen in FIG. 24A, the graphical user interface 760 for a device designed for a hospital setting can display a selection menu where a specific type of infection is selected. Such a device can be used with different removable chips, where each chip is configured to detect a particular type of infection by way of its sensing element (e.g., a chip for detecting Salmonella bacteria would include antibodies specific for Salmonella in the sensing element, and so on). In another embodiment, as seen in FIG. 24B, the graphical user interface 760 can display the raw impedance versus frequency curve generated from the sample.

The term “target analyte” as used herein generally refers to any molecule that is detectable with a biosensor as described herein. Non-limiting examples of targets that are detectable by the biosensor described herein include, but are not limited to, bacteria, viruses, proteins, nucleic acids, microRNAs, carbohydrates, and other types of small molecules that may indicate the presence of an infection, a cancer, or toxic analyte. Target analytes may also be, for instance, heavy metals.

It is to be understood that the target analytes that can be detected using the biosensor device described herein can be present in a sample that comprises tissue or fluid of a living organism. Non-limiting examples of tissue include soft tissue, hard tissue, skin, surface tissue, outer tissue, internal tissue, a membrane, fetal tissue, and endothelial tissue. The living organism can be a mammal and can include pet animals, such as dogs and cats; farm animals, such as cows, horses, and sheep; laboratory animals, such as rats, mice, and rabbits; poultry, such as chicken and turkeys; and, primates, such as monkeys and humans. In one embodiment, the mammal is human. It is also to be understood that the sample can comprise, for example, a surgical incision, an open wound, a closed wound, an organ, skin, skin lesions, membranes, and in situ fluids such as blood, urine, and the like.

In other embodiments, the sample can be a food source that could be contaminated by toxic organisms. Non-limiting examples of food sources can be grains, beverages, milk, and dairy products, fish, shellfish, eggs, commercially prepared and/or perishable foods for animal or human consumption (e.g., ground meat, salads, and the like). The sample can also be food tissue such as a fruit, an edible plant, a vegetable, a leafy vegetable, a plant root, a soy product, dead animal tissue, meat, fish, and eggs, where the presence of the target analyte is indicative of spoilage. In other embodiments, the sample can be in an external environment, such a soil, water ways, sludge, commercial effluent, and the like.

In some embodiments designed to detect bacteria, the presence of bacteria is detected as the bacterial antigens are bound to the antibodies. As a result of this interaction, the electrochemistry on the electrode changes. The rate and degree of change in the signal can be detected through one of several different methods. In one embodiment, where amperometric sensing is conducted, the current change due to the bacteria-antibody interaction is transmitted through the electrode. In another embodiment, where impedance sensing is conducted, the impedance variation in the electrode is measured.

The biosensor device may be designed to detect any specific bacteria that may cause infection by incorporating antibodies specific to the bacteria into the sensing matrix. Though certain embodiments described herein comprise antibodies specific for Staphylococcus aureus, the biosensor device can be also designed to detect any Gram-positive or Gram-negative bacteria, and rapidly differentiate between the two. By way of non-limiting example, antibodies specific for bacteria such as methicillin-resistant Staphyloccus aureus (MRSA), Staphylococcus epidermis, Staphylococcus saprophyticus, Streptococcus pyogenes, Streptococcus pneumoniae, Streptococcus agalactiae, Escherichia coli, Legionella pneumophila, Pseudomonas aeruginosa, Enterococcus faecalis, Listeria, Cyclospora, Salmonella enteritidis, Helicobacter pylori, Tubercle bacillus (TB), other Bacillus, Clostridium botulinum, Clostridium difficile, Clostridium perfringens, Clostridium tetani, Sporohalobacter, Anaerobacter, Heliobacterium, Brucella abortus, Brucella canis, Brucella melitensis, Brucella suis, Cyanobacteria, green sulfur bacteria, Chloroflexi, purple bacteria, thermodesulfobacteria, hydrogenophilaceae, nitrospirae, Burkholderia cenocepacia, Mycobacterium avium, Mycobacterium leprae, Mycobacterium tuberculosis, Mycobacterium ulcerans, Lactobacillus, Lactococcus, Bordetella pertussis, Chlamydia pneumoniae, Chlamydia tracomatis, Chlamydia psittaci, Borrelia burgdorferi, Campylobacter jejuni, Francisella tularensis, Leptospira monocytogenes, Leptospira interrogans, Mycoplasma pneumoniae, Rickettsia rickettsii, Shigella sonnei, Traponema pallidum, Vibrio cholerae, Haemophilus influenzae, Neiserria meningitidis, or Yersinia pestis can be incorporated into the sensing matrix, thereby enabling the biosensor to detect and/or quantify any such bacteria.

The biosensor device, as described herein, has applications in human treatment, veterinary care of animals, sampling of food sources, determination of the presence of pathogens in an external environment, and the like. There is a need for rapid, accurate, and affordable methods to detect the presence of pathogens in a wide variety of situations and circumstances, and the biosensor device is capable of supplying this need. In some embodiments, the biosensor device directly detects a pathogen. In other embodiments the biosensor device detects the antibodies, or immune response, to a pathogen.

The biosensor is useful for detecting infections in animals. Some examples of serious pathological agents in felines include, but are not limited to, Bartonella henselae, Borrelia burgdorferi, Chlamydia psittaci, Dirofilaria immitis, Ehrlichia canis, Feline Calicivirus, Feline Coronavirus, Feline Herpesvirus, Feline Immunodeficiency Virus, Feline Leukemia Virus, Leptospira spp., Mycoplasma haemofelis, Panleukopenia Virus, Toxoplasma gondii, and West Nile Virus.

Canine pathogens include, but are not limited to, Canine Adenovirus, Canine Distemper Virus, Canine Herpesvirus, Bordetella bronchiseptica, Neospora Hughesi and Caninum, Anaplasma phagocytophilum, Rickettsia rickettsii, Anaplasma platys, Canine parainfluenza virus, Tritrichomonas foetus, Clostridium difficle, Cryptosporidium spp., Cryptosporidium felis, Mycobacterium spp., Salmonella spp., Giardia spp., and Taenia spp.

Equine pathogens include, but are not limited to, Equine Herpes Virus, Equine Influenza A, Lawsonia intracellularis, Streptococcus equi, Equine Arteritis virus, Campylobacter jejuni, E. Coli, Shigella spp., Yersinia enterocolitica, Rhodococcus equi, West Nile and Leptospira spp.

Marine mammal pathogens include, but are not limited to, bacteria: Staph sp., Strep sp., Erysipelas rhusiopathiae, Bartonella, Coxiella, Chlamydia, Pseudomonas spp., Pseudomonas pseudomallei, Pseudomonas mallei, Klebsiella, E. coli, Salmonella sp., Clostridia perfringens and Enterococcus; viruses: Dolphin pox, seal pox, papilloma universal, papilloma manatee, canine adenovirus, influenza A and B, hepatitis A and B, Bovine enterovirus, Cosackivirus, encephalomyocarditis virus, Morbilliviruses, canine distemper virus, Bovine corona virus, Bovine rotavirus, universal herpes, and echovirus; fungi: Aspergillus, Nocardia, Histoplasma, Blastomyces, Coccidioides immitis, Lacazia loboi, Saksenaea, and Aphophysomyces.

Some examples of other analytes that can be detected include pesticides and/or toxins, such as: aflatoxins, arsenic, botulin, ciguatera toxin, cyanide, deoxynivalenol, dioxin, fungi, fumonisins, fusarium toxins, heavy metals, histadine, histamine, lead, marine toxins, mercury, mycotoxins, neurotoxin, nicotine, ochratoxin A toxins, patulin toxins, polychlorinated phenyls, pyrrolizidine alkaloids, ricin, scombrotoxins, shellfish toxin, tetrodotoxin, trichothecenes, zearelenone, and the combinations thereof.

Other target analytes may include food allergens, such as: almond, egg, gliadin, gluten, hazelnut, milk, peanut, soy residues, and combinations thereof.

In certain embodiments, the biosensor device can detect analytes over a desired time duration. The duration can be a first pre-determined time interval and at least a second pre-determined time interval that are calculated. In certain embodiments, an analyte correlation value is calculated during the test time interval.

Though a change in impedance is described herein for illustrative purposes, it is understood that depending on the particular embodiment, the biosensor device can utilize any of several principles of detection. In certain other embodiments, the types of signals detected include electrochemical (based on electrical properties), photometric (based on light properties), calorimetric (based on temperature change), and piezoelectric (based on elastic deformation of crystals caused by electrical potential).

The biosensor device may also be adapted for use in and/or incorporated into a variety of medical instruments or surgical tools, including but not limited to: endoscopic imaging devices, harvesting devices, retractors such as Hohmann retractors, bone hooks, skin hooks, nerve hooks, tension devices, forceps, elevators, drill sleeves, osteotomes, spinal rongeurs, spreaders, gouges, bone files and rasps, bone awls, rib shears, trephines, suction tubes, taps, tamps, calipers, countersinks, suture passers, and probes.

The biosensor device can deliver instantaneous, accurate sensing of a target analyte. In certain embodiments, the biosensor device can be fitted on a medical instrument adapted to check a human throat for the presence of Streptococcus. The biosensor device can be used by a physician or other medical personnel to determine whether a patient, such as a child patient, has a streptococcus infection by placing the tip of a medical instrument that includes the biosensor into the throat of the patient. In other embodiments, the biosensor device can be adapted for use in a hip revision procedure, wherein a medical instrument comprising the biosensor device is inserted to check for an infection such as tuberculosis of bone. The biosensor device enables immediate infection detection in any part of the body without having to wait for cultures.

In intraoperative procedures, a method that can sense the infection leading to determination of the following procedure does not exist to date. For example, in hip surgery, the current method still does not give determination of infection. The aspiration of the hip joint has to be shipped to a medical laboratory for evaluation. It will also involve an additional procedure to the patient. Under such circumstances, the surgeon can apply the sensor for the first reading while opening the hit joint for implantation. The second reading can be taken after the implant has been removed. This is the major area where the infection can be present. Use of the biosensor device aids in determining if a temporary implant with antibiotic administration needs to be applied after a wash out or a definite implantation can be done.

In clinical practice, for out-patient procedures, the infection sensor can be directly brought into contact with infected sites, and the outcome can be read on the display immediately. In clinical practice, for out-patient procedures, the sensor can be used to determine the pathogen on the swab of the infected area. In day care and clinical practice, it is a standard procedure to take the aspiration of the joint for evaluation. The fluid can be exposed to the sensor on a specially designed syringe device or applied on the biosensor device.

In some emerging economies, such as in Southeast Asia, a substantially amount of patients have tuberculosis. The biosensor device is especially useful as a non-invasive instrument to determine the presence of tuberculosis infections in real-time.

### EXAMPLES

Certain embodiments of the present invention are defined in the Examples herein. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

### Example 1

Cyclic voltammetry was used for electrochemical characterization of the sensing matrix described herein. Cyclic voltammetry is an electrochemical technique based on electrical current measurement as a function of voltage. The technique involves a working electrode where redox reactions or adsorption occurs, a reference electrode as a constant potential reference, an auxiliary or counter electrode that completes the circuit, an electrolyte, and a potentiostat (voltage source).

Gold circuits deposited on a micro interdigitated electrode acted as a transducer. The sensing matrix comprised a SAM and was formed on a gold electrode as the working electrode. The working electrode, a reference electrode, and a counter electrode were placed in a glass flask that was filled with electrolytes. Voltage was changed at a pre-determined rate and range, and the corresponding current change was recorded.

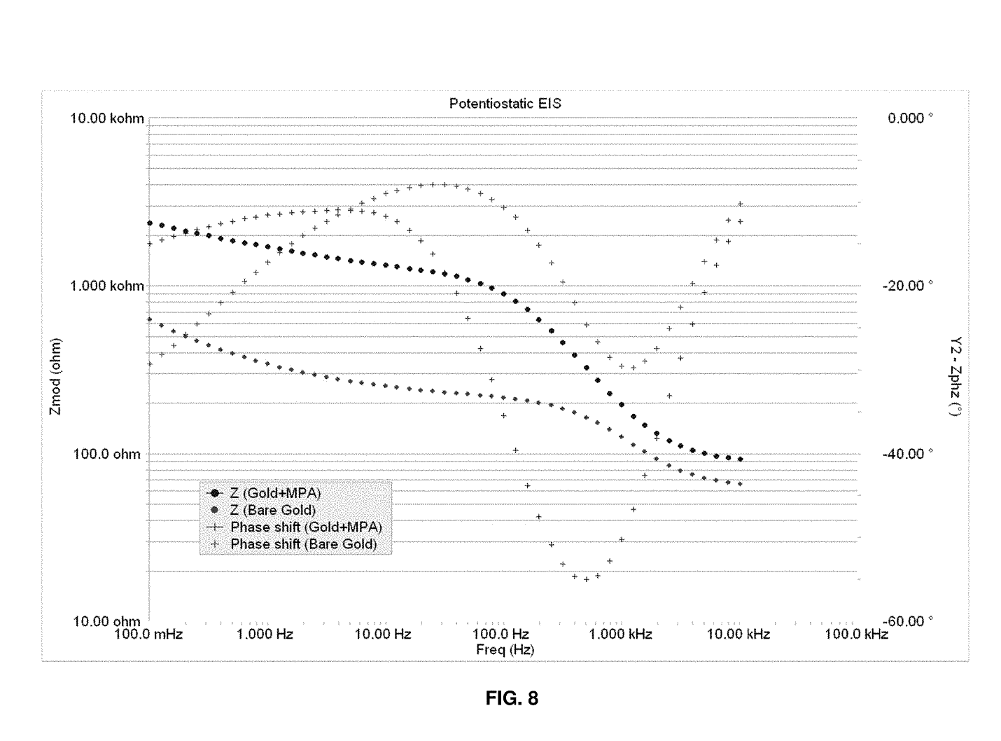

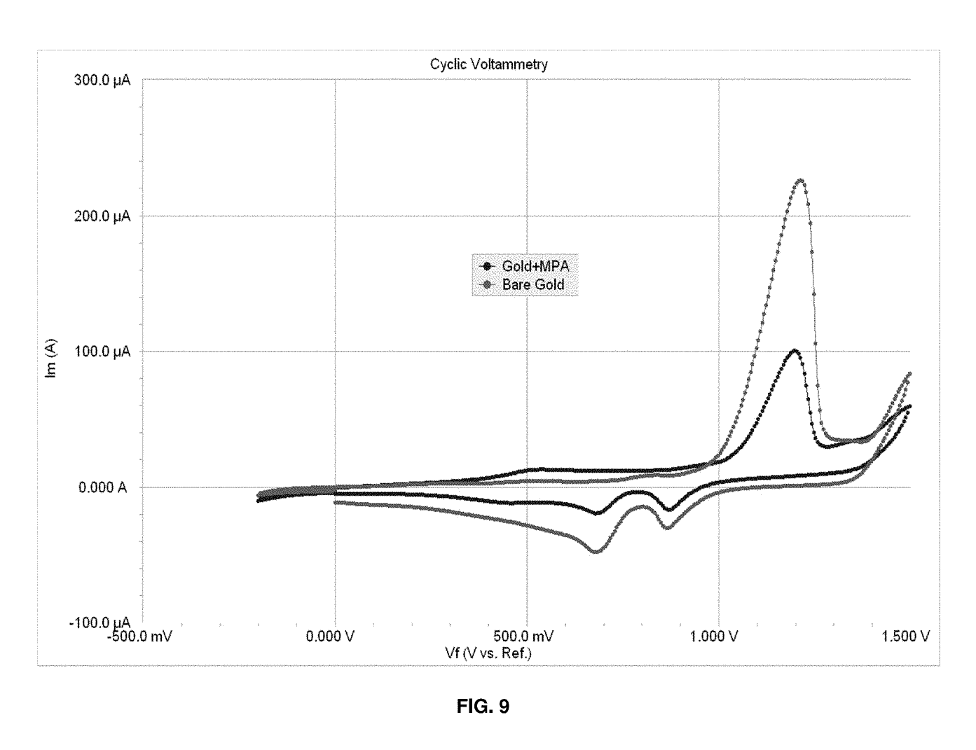

The gold electrode with SAM was shown to have higher impedance than a bare gold electrode. The gold electrode with MPA SAM was shown to have higher impedance magnitude and a different phase shift than the bare gold electrode. These results are depicted in the impedance curves in FIG. 5 and FIG. 8. Cyclic voltammetry revealed that a bare gold electrode has higher maximum current (lower resistance) than a gold electrode with MPA SAM. The cyclic voltammogram showing this is depicted in FIG. 9.

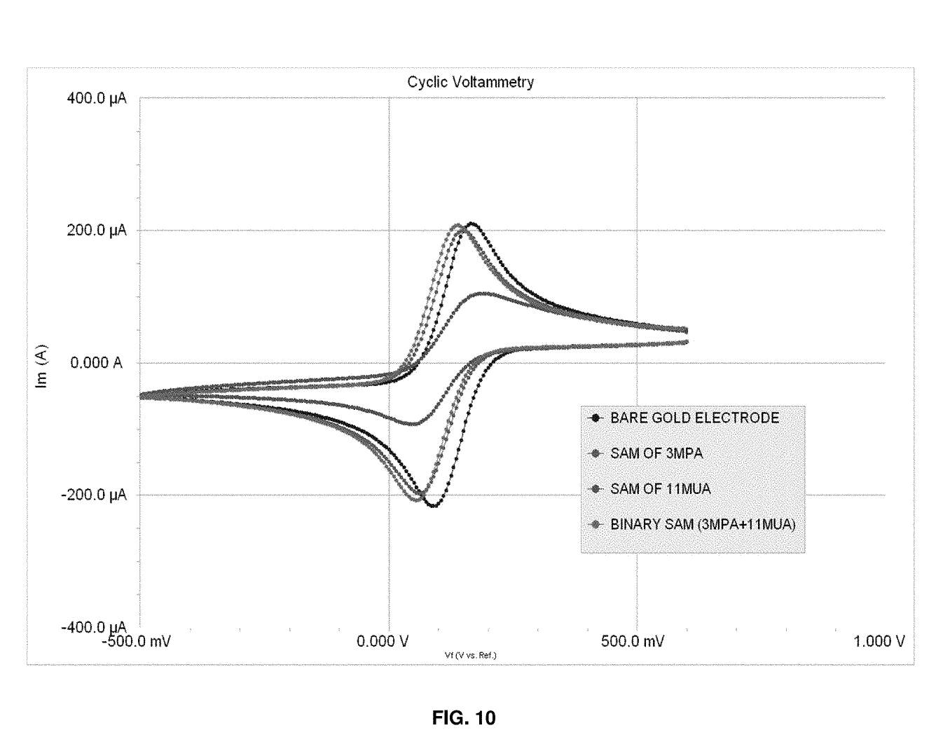

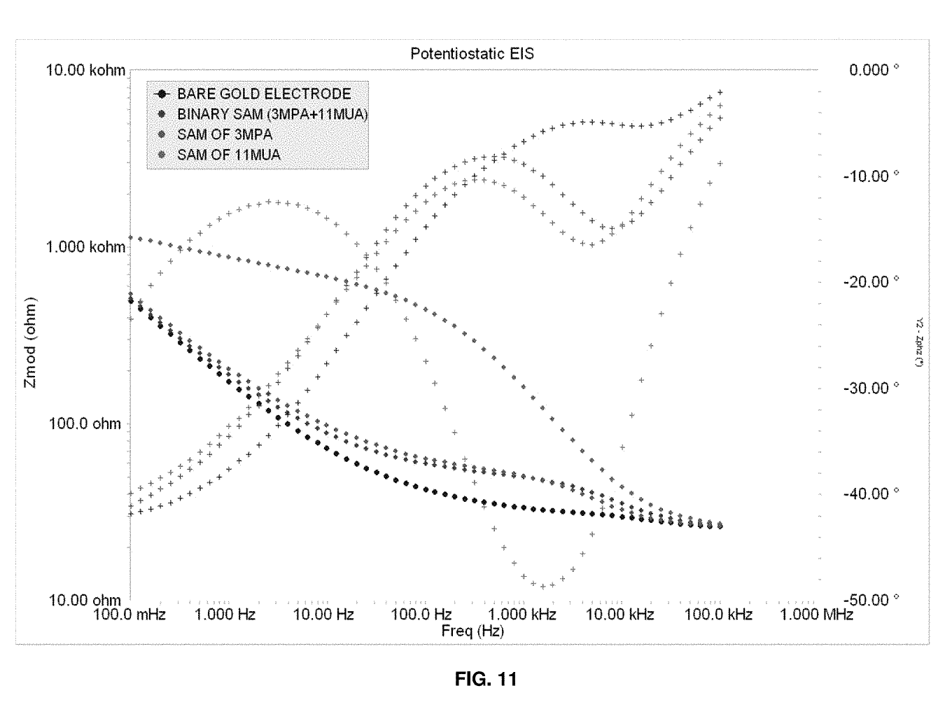

Different SAMs were tested. Specifically, four electrodes were compared: a bare gold electrode, a gold electrode with 3-MPA SAM, a gold electrode with 3-MPA and 11-MUA SAM, and a gold electrode with 11-MUA SAM. The gold electrode with 11-MUA SAM had not only the highest resistance, but also the highest impedance, and the most different phase shift trend. FIG. 10 and FIG. 11 show these results.

### Example 2

Screen printed electrodes (SPE) were sonicated in ethanol (99.5%) for 10 minutes and dried in a desiccator. A SPE was connected to a potentiostat and immersed in a conditioning solution containing 1 mL ammonium acetate buffer in 10 mL H₂O. Potential sweeping was performed from 0.6 V to −0.5 V for electrochemical conditioning of the gold electrode surface.

A self-assembled monolayer (SAM) was formed on the SPE gold surface. SPEs were soaked in a solution of 1 mM 11-mercaptoundecanoic acid (MUA) in ethanol for 12 hours and then rinsed with ethanol to remove unbounded 11-MUA molecules. The electrodes were then treated in a solution of 0.05 M 1-ethyl-3-(3-dimethylaminopropyl)carbodiimide (EDC) and 0.2 M N-hydroxysuccinimide (NHS) crosslinkers. After being rinsed and dried, a solution of 20 μg/mL of Staphylococcus antibody in a phosphate buffer solution (pH 7.2) was dropped on the electrode surface and then held still for 2 hour. The electrode was then rinsed with a phosphate buffer. In order to decrease non-specific adsorption, a solution of bovine serum albumin (BSA) in the phosphate buffer was used to block unreacted sites of the SAM.

### Example 3

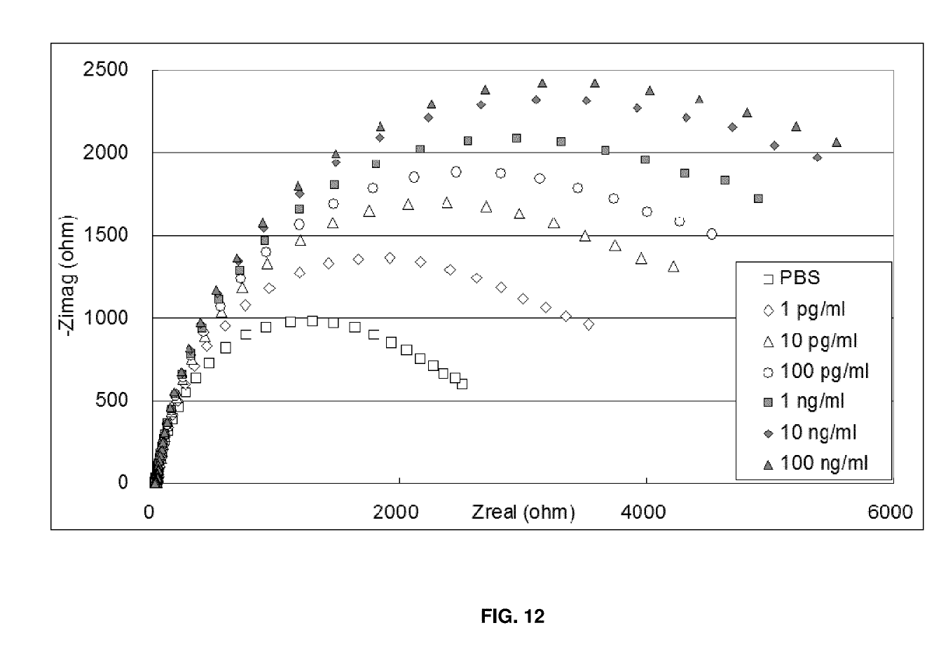

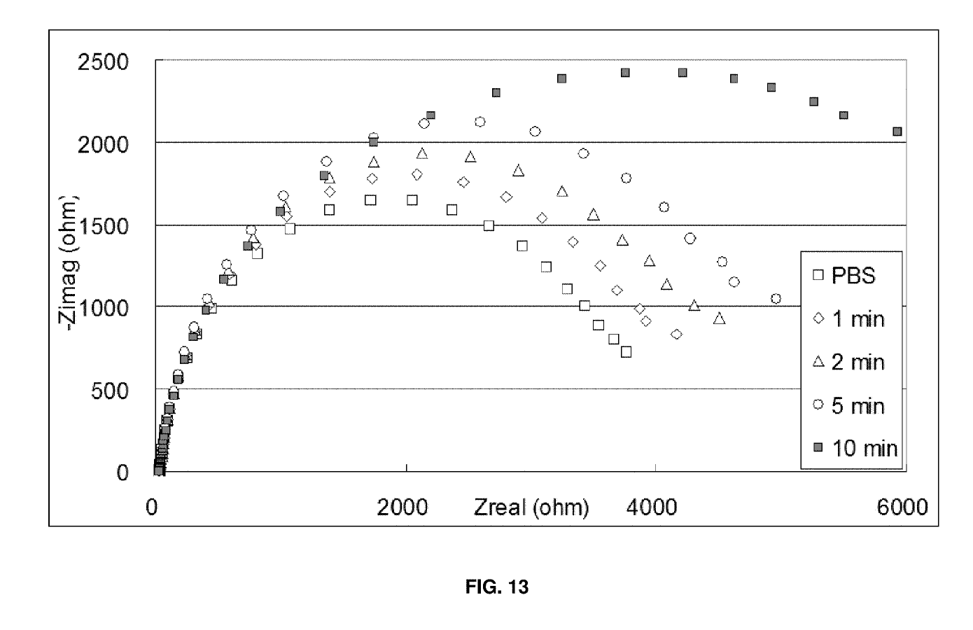

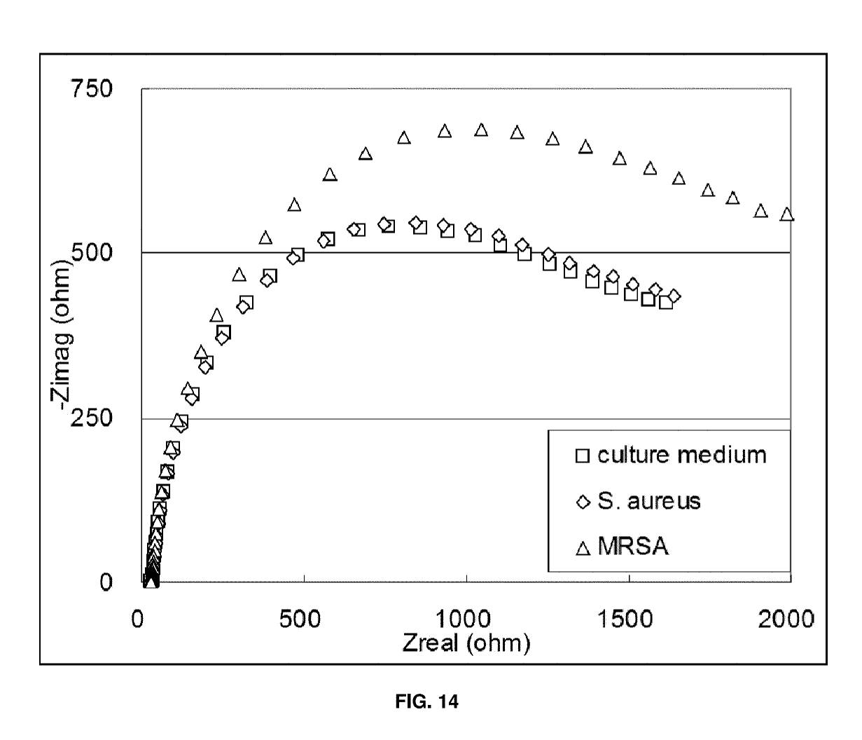

Electrochemical impedance spectroscopy (EIS) was performed using the software interface of the potentiostat from 1 Hz to 100 kHz. FIGS. 12-15 show plots of impedance versus frequency. FIG. 12 shows impedance curves that were generated by the sensing matrix comprising 11-MUA/MRSA antibody when it was exposed to serial dilutions of purified methicillin-resistant Staphylococcus aureus (MRSA) specific protein PBP2a in PBS for 10 minutes. The impedance shift was detectable at as low as 1 pg/ml of the protein, thus showing the sensitivity of this embodiment. FIG. 13 shows the responding time of the sensing, where the signal can be detected as rapidly as in 1 minute after the sensor exposed to the target protein. FIG. 14 shows an impedance curve generated by the sensing matrix comprising 11-MUA/MRSA antibody when exposed to the culture of 10⁶ cells/ml MRSA, 10⁶ cells/ml non-resistant Staphylococcus aureus, or blank culture medium. A significant shift was observed when MRSA was present.

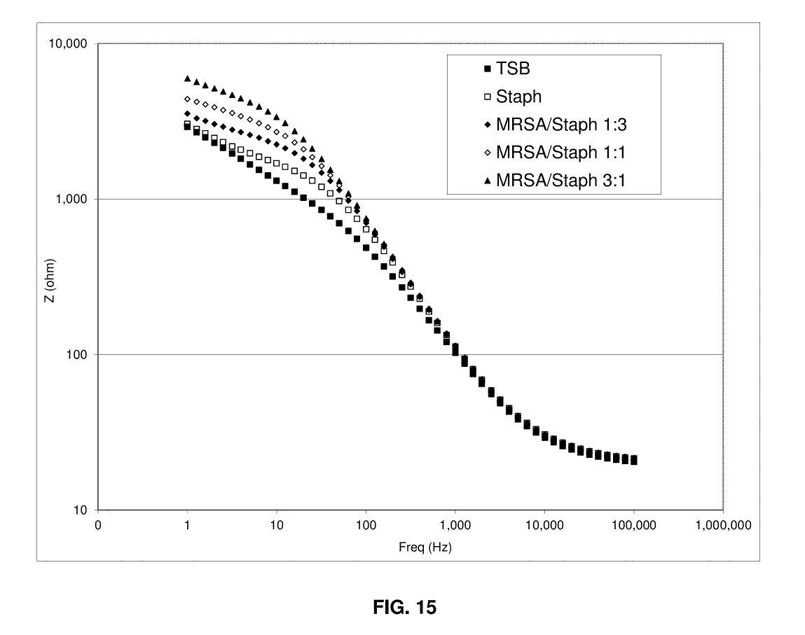

As shown in FIG. 15, when put in contact with the culture of 10⁶ cells/ml MRSA, 10⁶ cells/ml non-resistant Staphylococcus aureus, or blank culture medium, there was a significant shift was observed only when MRSA was present. Furthermore, as shown, in FIG. 15, this sensing method can specifically identify MRSA in a mixture of MRSA and the non-resistant strain. The shift of the curves corresponded to increased MRSA in the solution.

Although a significant change in impedance was not seen within one minute of putting the chip into a bacteria sample, the slope of the impedance-frequency (Z-f) curve changed immediately when MRSA bacteria were present. Thus, the Z-f curve slope, rather than the impedance magnitude itself, can be used as the sensing signal for fast detection.

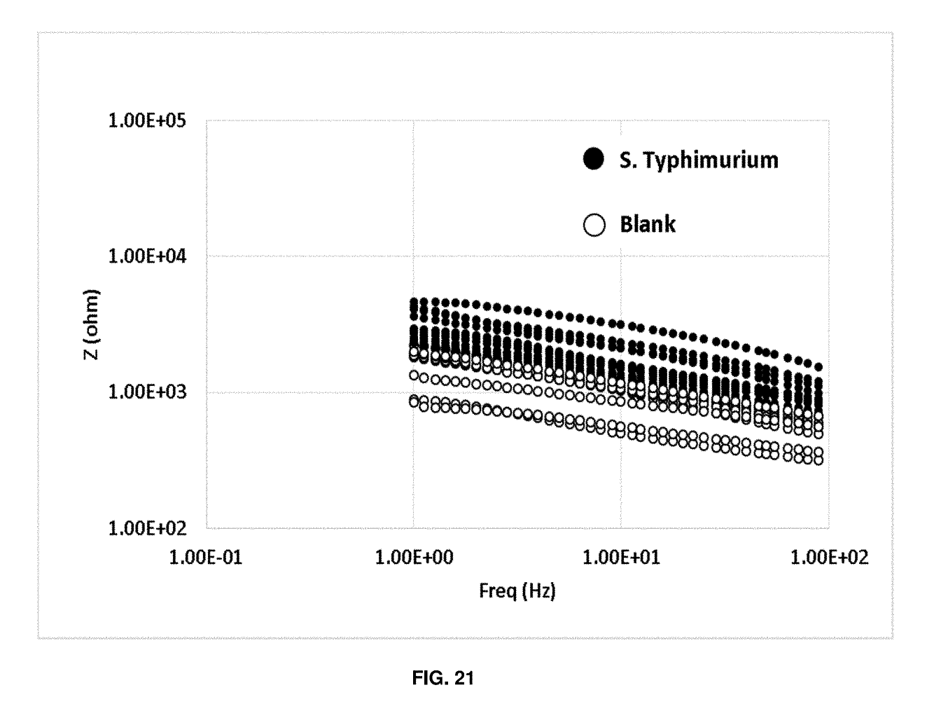

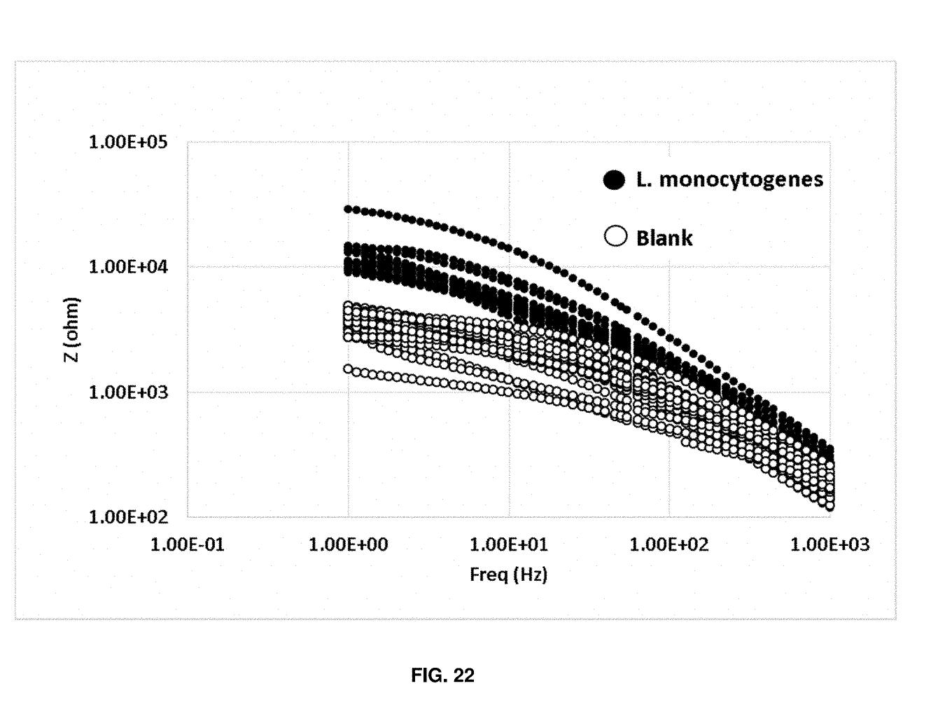

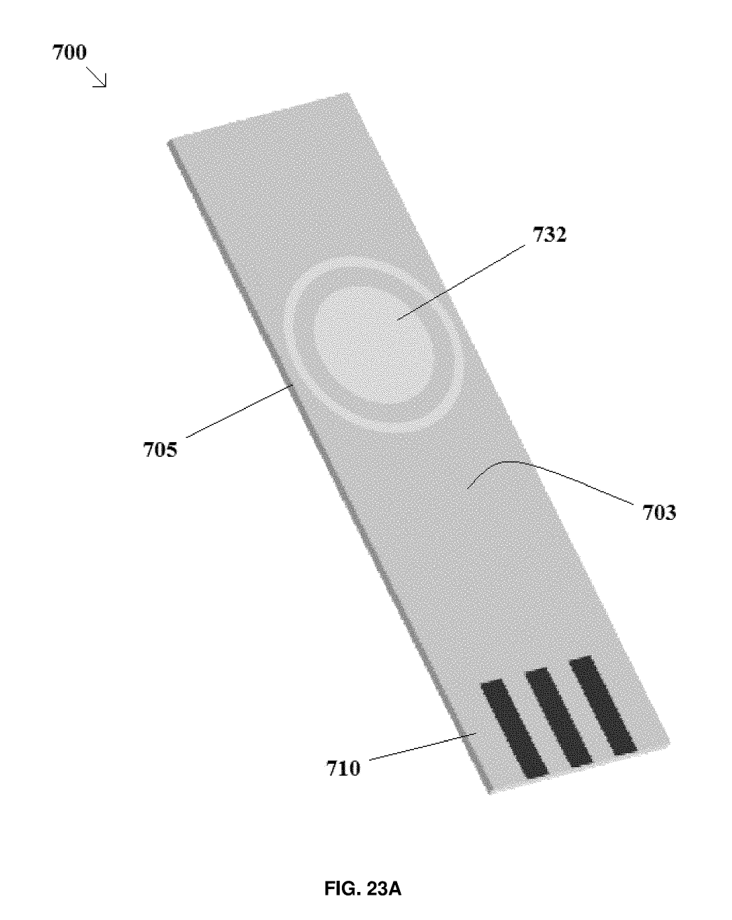

Similar tests were run with Salmonella and Listeria bacteria. Sensors for Salmonella bacteria were developed using anti-S. typhimurium LPS antibody (Abcam, Cambridge, Mass.) as the sensing element. Bacterial cultures were prepared by inoculating S. typhimurium (ATCC, Manassas, Va.) in nutrient broth, harvested after 16 hours, and diluted to 10⁵ CFU/ml. Fresh nutrient broth without bacteria served as control (blank) samples. For Listeria, sensors were developed using anti-Listeria antibody (RayBiotech, Norcross, Ga.) as the sensing element. Bacterial cultures were prepared by inoculating L. monocytogenes (ATCC, Manassas, Va.) in nutrient broth, harvested after 16 hours, and diluted to 10⁵ CFU/ml. Fresh nutrient broth without bacteria served as control (blank) samples. FIGS. 21-22 show the resulting impedance curves as a function of frequency, where FIG. 21 shows the results for Salmonella, and FIG. 22 shows the results for Listeria.

### Example 4

A biosensor with an anti-fouling agent was tested. A binary self-assembled monolayer consisting of 11-mercaptoundecanoic acid (11-MUA) and 3,6-dioxa-8-mercaptooctan-1-ol (DMOL) was formed on a gold coated surface. The surface was soaked in the solution of 11-MUA and DMOL in ethanol solution (99.5+%) for 12 hours and then rinsed with ethanol to remove unbounded molecules. Next, the surface was treated with a solution of 0.05 M 1-ethyl-3-(3-dimethylaminopropyl)carbodiimide (EDC) and 0.2 M N-hydroxysuccinimide (NHS). After being rinsed and dried, a solution of 20 μg/ml of Staphylococcus antibody in buffer solution was dropped on the surface and then held still for 1 hour. Through this process, the antibodies were attached to the top of 11-MUA. The surface was then rinsed with phosphate buffer. Finally, the surface was treated with a solution of BSA (bovine serum albumin) in a phosphate buffer solution to occupy the unreacted 11-MUA molecules.

EIS tests were performed using the software interface of the potentiostat from 1 Hz to 100 kHz, and impedance was plotted versus frequency. First, a blank culture media was tested. Then, the electrode was immersed in a MRSA bacteria solution and the EIS was tested after 30 min and 90 min. The electrode was washed and tested again. Then, the same electrode was tested with the regular Staphylococcus. No significant change was observed between the previous curve generated with MRSA after washing the sensor electrode. This indicates that the antibody was specific to MRSA.

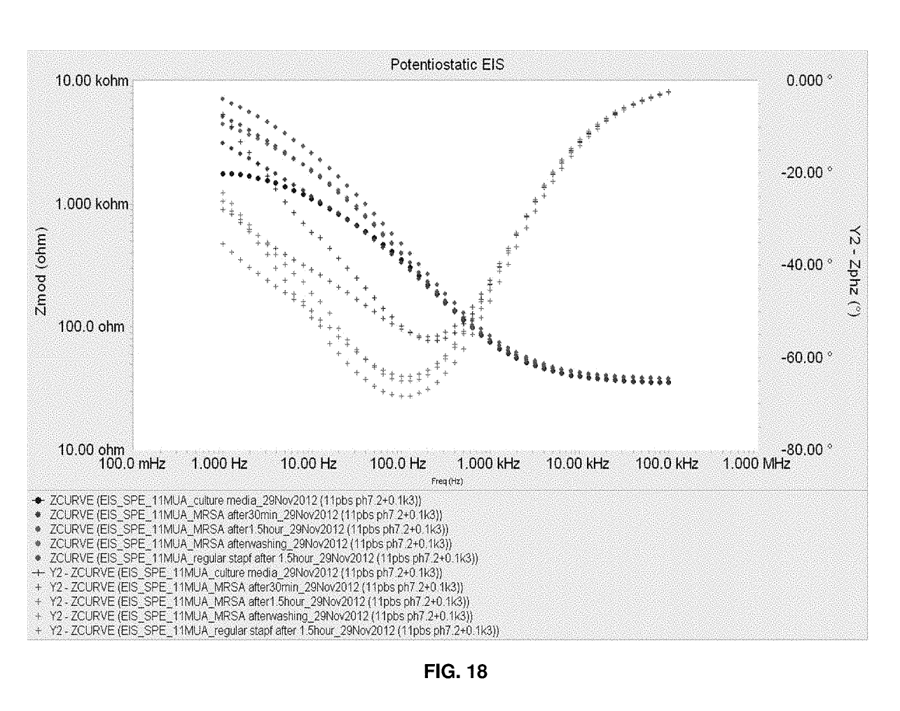

The impedance began increasing when the sensor electrode was put into contact with the bacteria, but after 1.5 hours, the impedance became constant. This indicates that after 1.5 hours, all the antibody sites were occupied by the bacteria. The EIS graph shown in FIG. 18 shows that the sensor is more sensitive at low frequencies. As seen from the EIS results in FIG. 18, the slope and magnitude changed during sensing due to the increasing binding of bacteria.

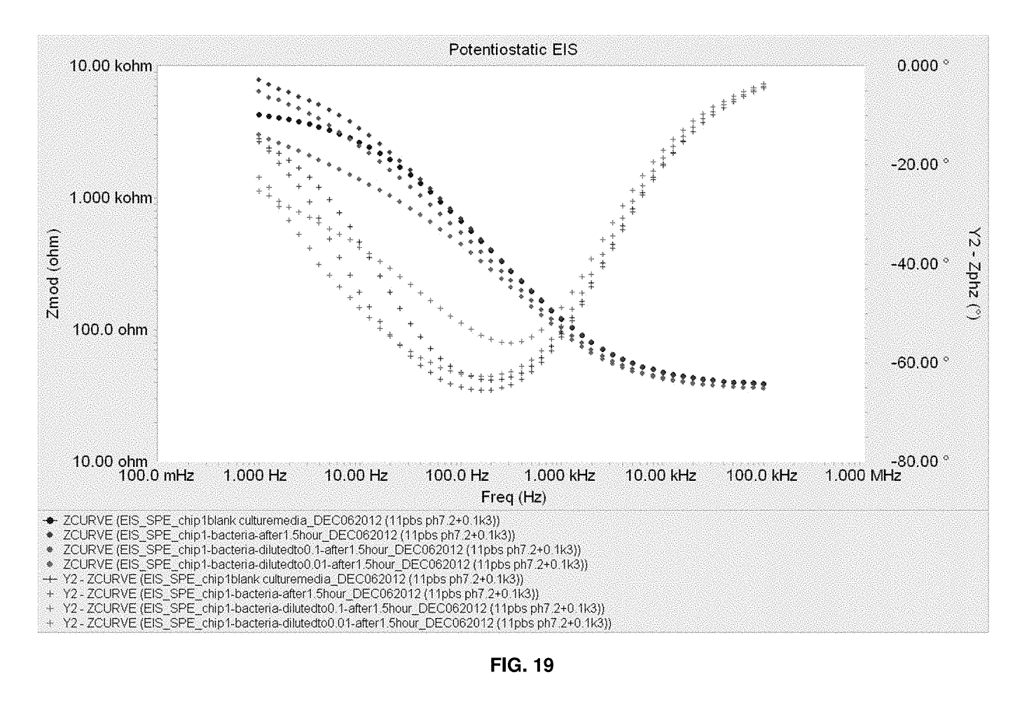

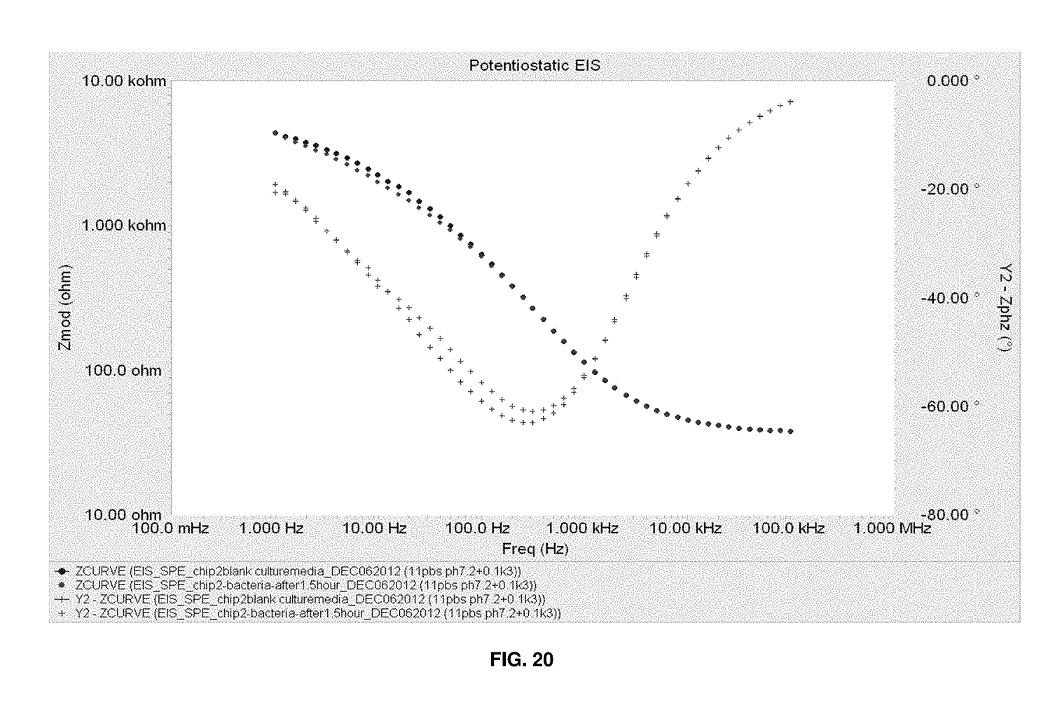

The bacteria concentration change was also investigated. The graph in FIG. 19 shows the results for the blank culture media, the MRSA bacteria sample that had the same cell concentration as in FIG. 18, a 10-fold diluted bacteria solution, and a 100-fold diluted bacteria solution. As seen in FIG. 19, when the bacteria solution is diluted, impedance decreases. A test with regular Staphylococcus was also conducted. The graph in FIG. 19 shows that the curve for blank culture media is very close to the regular Staphylococcus curve. This indicates that the antibody was specific to the MRSA.

Although a significant impedance change was not seen within one minute after putting the sensor into the bacteria samples, the slope of the Z-f curve changed immediately when MRSA bacteria was present. Therefore, for fast detection, Z-f curve slope can be used as a sensing signal rather than the impedance magnitude itself.

Certain embodiments and uses of the biosensor device and methods disclosed herein are defined in the examples herein. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

1. A biosensor for detecting the presence of a target analyte in a sample, the biosensor comprising:
a transducer component comprising a set of electrodes operatively connected to a microprocessor, the microprocessor being adapted to receive, process, and transmit a signal, wherein the set of electrodes includes at least one working electrode; and,
a receptor component having:
a sensing element capable of detecting and binding to at least one target analyte present in a sample, wherein the sensing element is attached to the working electrode either directly or via a self-assembled monolayer (SAM) layer positioned between and in contact with the sensing element and the working electrode; and
at least one anti-fouling agent linked to the working electrode;
the transducer component and the receptor component being capable of being brought into direct contact with the sample in situ,
wherein the sensing element, in the presence of the target analyte present in the sample, causes a detectable signal capable of being transmitted to the working electrode.

2. The biosensor of claim 1, wherein the anti-fouling agent comprises 3,6-dioxa-8-mercaptooctan-1-ol (DMOL) or PEG3.

3. The biosensor of claim 1, wherein the SAM layer comprises 11-mercaptoundecanoic acid (11-MUA) and 3,6-dioxa-8-mercaptooctan-1-ol (DMOL).

4. The biosensor of claim 1, wherein the SAM layer consists essentially of 11-mercaptoundecanoic acid (11-MUA) and 3,6-dioxa-8-mercaptooctan-1-ol (DMOL).

5. The biosensor of claim 1, wherein the sensing element comprises one or more proteins, peptides, nucleic acids, or polymers.

6. The biosensor of claim 1, wherein the sensing element comprises one or more of an antibody, an enzyme, an antibody fragment, DNA, RNA, an aptamer, an oligonucleotide, or a synthetic or natural polymer.

7. The biosensor of claim 1, wherein the sensing element comprises a polymer selected from the group consisting of alginate, chitosan, carboxymethyl cellulose, and derivatives thereof.

8. The biosensor of claim 1, wherein the sensing element is capable of detecting and binding to a heavy metal in the sample.

9. The biosensor of claim 1, wherein the biosensor is battery powered.

10. The biosensor of claim 1, wherein the SAM layer comprises mercaptoproprionic acid (MPA), 11-mercaptoundecanoic acid (MUA), 1-tetradecanethiol (TDT), or dithiobios-N-succinimidyl propionate (DTSP).

11. The biosensor of claim 1, wherein the microprocessor comprises a digital-to-analog converter and is programmed to generate an analog wave form, the biosensor further comprising an amplifier configured to attenuate the wave form to a desired level.

12-16. (canceled)

17. The biosensor of claim 1, wherein the sample comprises a fluid or tissue in a living organism in vivo.

18-31. (canceled)

32. The biosensor of claim 1, wherein the receptor component binds to a target analyte selected from the group consisting of: methicillin-resistant Staphyloccus aureus (MRSA), Staphylococcus epidermis, Staphylococcus saprophyticus, Streptococcus pyogenes, Streptococcus pneumoniae, Streptococcus agalactiae, Escherichia coli, Legionella pneumophila, Pseudomonas aeruginosa, Enterococcus faecalis, E. Coli, Listeria monocytogenes, Cyclospora, Salmonella enteritidis, Salmonella typhimurium, Helicobacter pylori, Tubercle bacillus (TB), other Bacillus, Clostridium botulinum, Clostridium difficile, Clostridium perfringens, Clostridium tetani, Sporohalobacter, Anaerobacter, Heliobacterium, Brucella abortus, Brucella canis, Brucella melitensis, Brucella suis, Cyanobacteria, green sulfur bacteria, Chloroflexi, purple bacteria, thermodesulfobacteria, hydrogenophilaceae, nitrospirae, Burkholderia cenocepacia, Mycobacterium avium, Mycobacterium leprae, Mycobacterium tuberculosis, Mycobacterium ulcerans, Lactobacillus, Lactococcus, Bordetella pertussis, Chlamydia pneumoniae, Chlamydia tracomatis, Chlamydia psittaci, Borrelia burgdorferi, Campylobacter jejuni, Francisella tularensis, Leptospira monocytogenes, Leptospira interrogans, Mycoplasma pneumoniae, Rickettsia rickettsii, Shigella sonnei, Traponema pallidum, Vibrio cholerae, Haemophilus influenzae, Neiserria meningitidis, and Yersinia pestis.

33-47. (canceled)

48. The biosensor of claim 1, wherein the biosensor wirelessly communicates a readout to a displaying system.

49. The biosensor of claim 48, wherein the displaying system is a smart phone or a tablet.

50-52. (canceled)

53. A biosensor for detecting the presence of a target analyte in a sample, the biosensor comprising:
a removable chip comprising a set of electrodes formed thereon and a connector end, wherein the connector end is configured to electrically connect the set of electrodes to a reader, the set of electrodes including a working electrode;
a sensing element capable of binding to at least one target analyte present in a sample, wherein the sensing element is attached to the working electrode either directly or via a self-assembled monolayer (SAM) layer positioned between and in contact with the sensing element and the working electrode; and
a processing device configured to receive a signal from the set of electrodes when the chip is connected to the reader, and display a graphical user interface;
wherein the sensing element, in the presence of the target analyte present in the sample, causes a detectable signal capable of being transmitted to the working electrode and received by the processing device.

54. The biosensor of claim 53, further comprising an anti-fouling agent attached to the set of electrodes.

55-56. (canceled)

57. A chip for a biosensor device, the chip comprising:
a support defining an elongated surface and having a connector end and a testing area;
a set of electrodes formed on the testing area of the surface, wherein the set of electrodes includes a working electrode; and
a sensing element linked to the working electrode either directly or via a SAM linker, wherein the sensing element is capable of binding to a target analyte;
wherein the sensing element, in the presence of the target analyte, causes a detectable signal capable of being transmitted to the working electrode.

58. The chip of claim 57, further comprising an anti-fouling agent on the set of electrodes.

59. The chip of claim 57, wherein the sensing element includes an antibody or other protein, a nucleic acid, a polymer, a peptide, or an aptamer.

60-68. (canceled)
